(19) United States
(12) Patent Application Publication
(10) Pub. No.: US 2023/0040069 A1
(43) Pub. Date: Feb. 9, 2023

(54) SEPARATING AROMATIC ISOMERS USING AQUEOUS SOLUTIONS OF CUCURBITURIL MACROCYCLES

(71) Applicant: King Abdullah University of Science and Technology, Thuwal (SA)

(72) Inventors: Gengwu ZHANG, Thuwal (SA); Niveen M. KHASHAB, Thuwal (SA)

(21) Appl. No.: 17/785,560

(22) PCT Filed: Dec. 17, 2020
(86) PCT No.: PCT/IB2020/062139

§ 371 Date: Jun. 15, 2022

Related U.S. Application Data
(60) Provisional application No. 63/027,395, filed on May 20, 2020.
(60) Provisional application No. 62/949,034, filed on Dec. 17, 2019.

(51) Int. Cl. C07C 7/11; C07B 63/00; C07C 17/38; B01D 11/04

(52) U.S. Cl. CPC C07C 7/11; C07B 63/00; C07C 17/38; B01D 11/0492; C07B 2200/09

(57) ABSTRACT

The present disclosure provide processes of separating aromatic isomers; liquid-liquid extraction systems and processes; liquid-liquid extraction solvents; cucurbituril macrocycle selective for the extraction of aromatic isomers; related materials, methods, and systems; and the like. The process of separating aromatic isomers may include contacting an isomers solution including one or more aromatic isomers, with an aqueous solution including a cucurbituril macrocycle, to produce a first aqueous phase and a first organic phase, wherein the cucurbituril macrocycle is selective for the extraction of at least one of said aromatic isomers.

### BACKGROUND

With the increasing demand on chemicals driven by the rapid development of industries, such as the petrochemical and pharmaceutical industries, innovative materials and technologies are essential to improve the efficiency, quality, and safety of these processes. Disubstituted benzene isomers such as xylenes, dichlorobenzenes, dibromobenzenes, chlorotoluenes and others are often used for many polymers, plastics, fibers, solvents and fuel. The production of these materials in industry results in mixed products. For example, a mixture of xylene isomers is obtained by catalytic reforming. Chlorotoluene isomers or dichlorobenzene isomers is obtained from chlorination of toluene or chlorobenzene. The isolation and purification of these isomers, among others, has been one of the most challenging separations to achieve, due to identical molecular weights, similarity in chemical structures, and small differences in boiling points. For example, xylene isomers have the same molecular weight (106 g/mol), similar boiling points (138° C. for p-xylene (PX), 139° C. for m-xylene (MX) and 144° C. for o-xylene (OX)), and similar kinetic diameters (6.7 Å for p-xylene, 7.1 Å for m-xylene and 7.4 Å for o-xylene).

Solvent extraction is used by refineries to separate a mixture of crude oil into pure benzene, toluene and mixed xylenes products. However, this strategy is not effective for separating disubstituted benzene isomers due to the similarity of their solubilities in most solvents. Currently, meta-xylene (MX) can be extracted by highly toxic and corrosive HF-BF3 which causes damage to equipment and, most importantly, is harmful to the environment. Distillation, which is the traditional separation method for benzene derivatives in industry, is not feasible due to the exorbitant numbers of theoretical plates required to achieve the separation and the correspondingly high energy costs. Fortunately, para-substituted benzene isomers have the highest melting point, which is around or above room temperature, while ortho- and meta-isomers have lower melting points. This indicates that para-isomers can be separated from ortho- and meta-by fractional crystallization or low temperature fractional crystallization. However, the crystallization process requires low temperatures or high pressures to achieve the eutectic point which drives up energy costs, while product recovery is merely 60-70%.

Selective adsorption by porous materials, such as zeolites, metal organic frameworks (MOFs), covalent organic frameworks (COFs), porous organic cages (POCs) and porous coordination polymers (PCPs) can be used for the separation of disubstituted benzene isomers. Nonporous adaptive crystals (NACs), which are mostly based on pillararenes, have achieved selective guest separation due to their unique pillar-like structure and extensive host-guest properties. However, in all the cases of solid-vapor adsorption separation, the vapor production and desorption processes require high temperatures and high pressures which means steep energy requirements. Furthermore, most porous materials only show high selectivity for a specific disubstituted benzene isomer because of the fixed pore size of the hosts. If a separation of a different isomer is required, the pore size needs to be modified or the whole material effectively replaced. Accordingly, although such porous materials can be used, they suffer from numerous disadvantages.

### SUMMARY

According to an aspect of the present invention, a process for separating aromatic isomers may include contacting an isomers solution including one or more aromatic isomers, with an aqueous solution including a cucurbituril macrocycle, to produce a first aqueous phase and a first organic phase, wherein the cucurbituril macrocycle is selective for the extraction of at least one of said aromatic isomers.

According to another aspect of the present invention, a process for separating aromatic isomers (e.g., a liquid-liquid extraction process) may include one or more of the following steps: providing an isomers solution including at least one aromatic isomer and further providing an aqueous solution including a cucurbituril macrocycle; extracting at least a portion of the aromatic isomer from the isomers solution, using the aqueous solution, to produce a first aqueous phase and a first organic phase, wherein the first aqueous phase includes at least a portion of the cucurbituril macrocycle from the aqueous solution and the portion of the aromatic isomer extracted from the isomers solution; recovering at least a portion of the aromatic isomer from the first aqueous phase, using an organic solution, to produce a second aqueous phase and a second organic phase, wherein the second aqueous phase includes at least a portion of the cucurbituril macrocycle from the first aqueous phase and wherein the second organic phase includes the portion of the aromatic isomer recovered from the first aqueous phase; and recycling and/or reusing the second aqueous phase for one or more extraction cycles.

According to a further aspect of the present invention, a liquid-liquid extraction solvents may include an aqueous solution of a cucurbituril macrocycle, wherein the cucurbituril macrocycle is selective for the extraction of at least one aromatic isomer and wherein the cucurbituril has the following chemical structure:

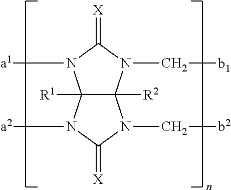

wherein:

a¹ indicates the point of attachment to b¹;

a² indicates the point of attachment to b²;

n is 1-20;

X is O, S, or NH; and

R¹ and R² are independently selected from the group consisting of hydrogen, H, optionally substituted C₁-C₃₀ alkyl group; optionally substituted C₂-C₃₀ alkenyl group; optionally substituted C₂-C₃₀ alkynyl group; optionally substituted C₂-C₃₀ carbonylalkyl group; optionally substituted C₁-C₃₀ thioalkyl group; optionally substituted C₁-C₃₀ alkylthiol group; optionally substituted C₁-C₃₀ hydroxyalkyl group; optionally substituted C₁-C₃₀ alkylsilyl group; optionally substituted C₁-C₃₀ aminoalkyl group; optionally substituted C₁-C₃₀ aminoalkylthioalkyl group; optionally substituted C₅-C₃₀ cycloalkyl group; optionally substituted C₂-C₃₀ heterocycloalkyl group; optionally substituted C₆-C₃₀ aryl group; optionally substituted C₆-C₃₀ arylalkyl group; optionally substituted C₄-C₃₀ heteroaryl group; and optionally substituted C₄-C₃₀ heteroarylalkyl group.

### BRIEF DESCRIPTION OF THE DRAWINGS

FIG. 1A is a flowchart of a liquid-liquid extraction process for separating aromatic isomers, in accordance with one or more embodiments of the present invention.

FIG. 1B is a flowchart of a liquid-liquid extraction process for separating aromatic isomers, in accordance with one or more embodiments of the present invention

FIG. 2 is a schematic diagram of a liquid-liquid extraction system for separating aromatic isomers, in accordance with one or more embodiments of the present invention.

FIG. 3 is a schematic diagram of another liquid-liquid extraction system for separating aromatic isomers, in accordance with one or more embodiments of the present invention.

FIGS. 4A-4E illustrate a separation process and presents results of xylene isomers separation by cucurbit[7]uril, showing in (A) a schematic illustration of the separation process; (B) ¹H NMR spectra (600 MHz, 298K, D₂O) of the water phase in the first extraction process (signals of aromatic protons of xylenes located between 6.0-7.6 ppm were scaled up in the dashed red line area); (C) partial ¹H NMR spectra (600 MHz, 298K, CDCl₃) of the organic phase in the second extraction process; (D) relative ratios of isomers after the separation from a 1:1:1 xylene mixture measured by gas chromatography; and (E) the percentage of PX, MX, and OX separated by CB7 within 5 recycles, in accordance with one or more embodiments of the present invention.

FIG. 5 presents results of host-guest experiments involving CB7 and xylenes, including from bottom to top ¹H NMR spectra (600 MHz, 298K, D₂O) of CB7; CB7 with 2 eq. OX; OX; CB7 with 2 eq. MX; MX; CB7 with 2 eq. PX; PX; and CB7 with 1.2 eq. xylene isomers (1:1:1, v:v:v) (where [CB7]=1.0 mM; aromatic protons of xylene molecules at 5.9-7.5 ppm was scaled up in the dashed red line area; methyl protons of xylene molecules at 1.3-2.6 ppm was scaled up in the dashed blue line area; and * means the complexed proton signals), in accordance with one or more embodiments of the present invention.

FIG. 6 is a 2D NOESY spectrum (950 MHz, 298K, D₂O) for the complex OX@CB7 (cross-peak signals between CB7 and OX were scaled up in the dashed red line area) ([CB7]=2.0 mM; [OX]=1.3 mM), in accordance with one or more embodiments of the present invention.

FIGS. 7A-7F present kinetic analysis of complexes between CB7 and xylenes, including variable-temperature ¹H NMR spectra (600 MHz, D₂O) of CB7 and (A) PX, (B) MX, (C) OX (the ratios of complexed and un-complexed xylene molecules were 1:1 determined by integration of methyl proton signals in xylenes; decomplexation rates were obtained by analyzing the slow exchange signals of methyl protons in xylenes (black trace, experimental; blue trace, simulated)) and Eyring plots of the variable-temperature data (red line, best linear fit) which were used to determine the kinetic parameters of the complexes (D) PX@CB7, (E) MX@CB7, (F) OX@CB7, in accordance with one or more embodiments of the present invention.

FIGS. 8A-8B provide schematic illustrations for the shape-fitting complexation process of CB7 and xylenes: (A) molecular size and shape of PX, MX, OX; and (B) schematic illustration for the shape-fitting complexation process of CB7 and xylenes in water, in accordance with one or more embodiments of the present invention.

FIG. 9 is a schematic diagram showing optimized structures and binding energies of various complexes of CB7@xylene, where top and side views of DFT optimized structure of the CB7 complexes with xylenes were calculated at ωB97XD/6-31G(d) level and binding energies in gas phase and water phase was calculated at ωB97XD/6-311+G(d) level (hydrogen, carbon, nitrogen and oxygen atoms are white, grey, blue and red, respectively), in accordance with one or more embodiments of the present invention.

FIG. 10 is a graphical view showing the relative xylene isomer ratio after separation of a 1:1:1 mixture of the three xylene isomers measured by using gas chromatography, according to one or more embodiments of the invention.

FIG. 11 is ¹H NMR spectra (600 MHz, CDCl₃, 298K) of organic phase after extraction with CB7 and GL aqueous solution, according to one or more embodiments of the invention.

FIG. 12 is 2D NOESY spectrum (950 MHz, 298K, D₂O) of the complex PX@CB7, according to one or more embodiments of the invention.

FIG. 13 is 2D NOESY spectrum (700 MHz, 298K, D₂O) of the complex MX@CB7, according to one or more embodiments of the invention.

FIGS. 14A-14B are graphical views of isothermal titration calorimetry (ITC) isotherms for the titration of CB7 (1.1 mM) into PX (58 μM) in aqueous solution at 25° C., according to one or more embodiments of the invention.

FIGS. 15A-15B are graphical views of isothermal titration calorimetry (ITC) isotherms for the titration of CB7 (1.1 mM) into MX (82 μM) in aqueous solution at 25° C., according to one or more embodiments of the invention.

FIGS. 16A-16B are graphical views of isothermal titration calorimetry (ITC) isotherms for the titration of CB7 (1.1 mM) into OX (133 μM) in aqueous solution at 25° C., according to one or more embodiments of the invention.

FIG. 17 is a graphical view showing the relative xylene isomer ratio after separation of a 1:1 mixture of PX/MX measured by using gas chromatography, according to one or more embodiments of the invention.

FIG. 18 is a graphical view showing the xylene isomer ratio after separation of a 1:1:1 mixture of CT isomers measured by using gas chromatography, according to one or more embodiments of the invention.

FIG. 19 is a graphical view showing the relative xylene isomer ratio after separation of a 1:1:1 mixture of DCB isomers measured by using gas chromatography, according to one or more embodiments of the invention.

FIG. 20 is a graphical view showing the relative xylene isomer ratio after separation of a 1:1:1 mixture of DBB isomers measured by using gas chromatography, according to one or more embodiments of the invention.

FIG. 21 is a graphical view showing the relative xylene isomer ratio after separation of a 1:1:1 mixture of CFB isomers measured by gas chromatography, according to one or more embodiments of the invention.

FIG. 22 is partial ¹H NMR spectra (600 MHz, D₂O, 298K) of CB7, and CB7 with PCFB, according to one or more embodiments of the invention.

FIG. 23 is partial ¹H NMR spectra (600 MHz, D₂O, 298K) of CB7, and CB7 with MCFB, according to one or more embodiments of the invention.

FIG. 24 is partial ¹H NMR spectra (600 MHz, D₂O, 298K) of CB7, and CB7 with different equivalent of OCFB, for separations provided in Example 9, according to one or more embodiments of the invention.

FIG. 25 is a schematic diagram of another liquid-liquid extraction system for separating trimethylbenzene isomers, in accordance with one or more embodiments of the present invention.

FIGS. 26A-26B are graphical views of (a) the ratio of trimethylbenzene isomers in the triisopropylbenzene (TIPB) phase and (b) the ratio of trimethylbenzene isomers in the organic phase, in accordance with one or more embodiments of the invention.

### DETAILED DESCRIPTION

### Definitions

The terms recited below have been defined as described below. All other terms and phrases in this disclosure shall be construed according to their ordinary meaning as understood by one of skill in the art.

As used herein, the term “isomers solution” and/or “isomers mixture” refers to a mixture/solution containing at least one aromatic isomer or, more typically, two or more aromatic isomers.

As used herein, the term “aromatic isomer(s)” refers one or more aromatic compounds (e.g., substituted aromatic compounds) having molecular formulas identical to at least one other aromatic compound, except with a different spatial arrangement of one or more atoms. Aromatic isomers may include benzene isomers (e.g., mono- and multi-substituted benzene isomers) and other substituted aromatic compounds. Although the term as used herein includes diastereomers (e.g., cis/trans isomers, conformers, and/or rotamers) as well as enantiomers, it more typically includes constitutional or positional isomers. Isomers of a substituted aromatic compound may include positional isomers, such as substituted benzene isomers. Positional isomers include isomers having identical chemical formulas but distinct and/or different chemical structures. For example, positional isomers may include isomers that differ from each other in the positioning of a substituent or functional group. Examples of positional isomers include, without limitation, one or more of ortho-substituted aromatic isomers, meta-substituted aromatic isomers, para-substituted aromatic isomers, other multi-substituted aromatic isomers (e.g., di-, tri-, tetra-, penta-, hexa-, etc. substituted aromatic isomers), mono-substituted aromatic isomers, and aromatic isomers having other substitution patterns. Examples of aromatic isomers having other substitution patterns include, without limitation, C₃-benzenes, such as for example trimethylbenzenes (e.g., 1,2,3-trimethylbenzene, 1,2,4-trimethylbenzene, and 1,3,5-trimethylbenzene, 1,2-ethylmethylbenzene, 1,3-ethylmethylbenzene, 1,4-ethylmethylbenzene, cumene, n-propylbenzene, and the like). Further examples of aromatic isomers are provided elsewhere herein and thus these shall not be limiting.

As used herein, the term “benzene isomer(s)” refers to one or more benzene compounds having molecular formulas identical to at least one other benzene compound, except with a different spatial arrangement of atoms. The term includes benzene isomers having two substituents (e.g., disubstituted benzene isomers), as well as benzene isomers having more than two substituents (e.g., multi-substituted benzene isomers), and in some instances mono-substituted benzene isomers. The term also permits, but does not require, the presence of heteroatoms in the ring structure of benzene, wherein at least one of the carbon atoms of the benzene ring is replaced with a heteroatom.

The term includes ortho-substituted benzene isomers having the chemical structure of formula (1A):

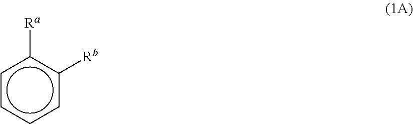

wherein R^{a} and R^{b} are independently halo, alkyl, alkoxy, hydroxyl, amino, alkylamino, cyano, isocyanate, nitro, vinyl, or allyl. Although not shown, the ortho-substituted benzene isomer of the formula (1A) can include from 1 to 4 additional substituents, which are defined below.

The term includes meta-substituted benzene isomers having the chemical structure of formula (1B):

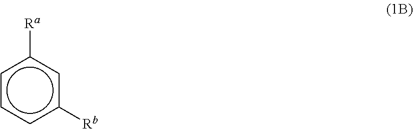

wherein R^{a} and R^{b} are as defined above for (1A). Although not shown, the meta-substituted benzene isomer of the formula (1B) can include from 1 to 4 additional substituents, which include those as defined herein.

The term includes para-substituted benzene isomers having the chemical structure of formula (1C):

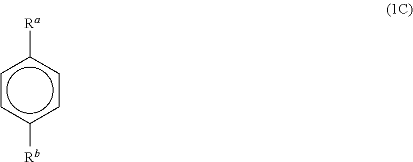

wherein R^{a} and R^{b} are as defined above for (1A). Although not shown, the para-substituted benzene isomer of the formula (1C) can include from 1 to 4 additional substituents, as defined herein.

As used herein, the term “cucurbituril” and/or “cucurbituril macrocycle” generally refers to a macrocyclic molecule having a central cavity and comprising glycoluril monomer units linked by methylene (—CH₂—) bridges. In some embodiments, the central cavity of cucurbituril is symmetric or substantially symmetric and/or hydrophobic. Unless provided otherwise, the term includes hydrates, salts, derivatives, and analogues of cucurbituril. For example, the term includes cucurbituril macrocycles having the chemical structure of formula (2):

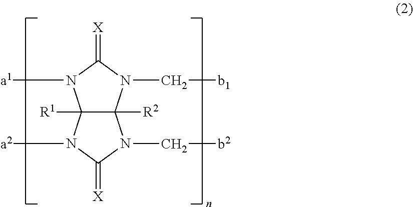

or hydrates, salts, and/or analogues thereof, wherein:

a¹ indicates the point of attachment to b¹;

a² indicates the point of attachment to b²;

n is from 1 to 20;

X is O, S, or NH; and

R¹ and R² are independently selected from the group consisting of hydrogen, optionally substituted C₁-C₃₀ alkyl group; optionally substituted C₂-C₃₀ alkenyl group; optionally substituted C₂-C₃₀ alkynyl group; optionally substituted C₂-C₃₀ carbonylalkyl group; optionally substituted C₁-C₃₀ thioalkyl group; optionally substituted C₁-C₃₀ alkylthiol group; optionally substituted C₁-C₃₀ hydroxyalkyl group; optionally substituted C₁-C₃₀ alkylsilyl group; optionally substituted C₁-C₃₀ aminoalkyl group; optionally substituted C₁-C₃₀ aminoalkylthioalkyl group; optionally substituted C₅-C₃₀ cycloalkyl group; optionally substituted C₂-C₃₀ heterocycloalkyl group; optionally substituted C₆-C₃₀ aryl group; optionally substituted C₆-C₃₀ arylalkyl group; optionally substituted C₄-C₃₀ heteroaryl group; and optionally substituted C₄-C₃₀ heteroarylalkyl group.

An unsubstituted cucurbituril refers to a cucurbituril in which R¹ and R² are both hydrogen in all the glycoluril units of formula (2). A substituted cucurbituril refers to a cucurbituril in which at least one of R¹ and R² is other than a hydrogen for at least one glycoluril unit of formula (2). A cucurbituril, whether substituted or unsubstituted, can be referred to herein as a “cucurbit[n]uril” or “CB[n]” where n represents the number of glycoluril monomer units present in the macrocycle. Examples of suitable cucurbiturils include, without limitation, the following: cucurbit[2]uril, cucurbit[3]uril, cucurbit[4]uril, cucurbit[5]uril, cucurbit[6]uril, cucurbit[7]uril, cucurbit[8]uril, cucurbit[8]uril, cucurbit[9]uril, cucurbit[10]uril, cucurbit[11]uril, cucurbit[12]uril, cucurbit[13]uril, cucurbit[14]uril, cucurbit[15]uril, cucurbit[16]uril, cucurbit[17]uril, cucurbit[18]uril, cucurbit[19]uril, cucurbit[20]uril, analogues thereof, and combinations thereof.

A cucurbituril can include substituted and unsubstituted glycoluril monomer units. Such cucurbiturils refer to a cucurbituril in which R¹ and R² are both hydrogen in at least one glycoluril unit of formula (2) and in which R¹ and R² is other than a hydrogen for at least one glycoluril unit of formula (2). Such cucurbiturils can be referred to herein as a “cucurbit[s,u]uril,” where s represents the number of substituted glycoluril monomer units and u represents the number of unsubstituted glycoluril monomer units.

As used herein, the term “host-guest complex” generally refers to a supramolecular assembly containing a macrocyclic host species and a guest species bound together via non-covalent interactions. Examples of non-covalent interactions include, without limitation, hydrophobic forces, electrostatic forces, van der Waals forces, hydrogen bonding, and the like. The macrocyclic host species is usually characterized by a cavity within which at least a portion of the guest species resides.

As used herein, the term “alkyl” refers to a straight- or branched-chain or cyclic hydrocarbon radical or moiety comprising only carbon and hydrogen atoms, containing no unsaturation, and having 30 or fewer carbon atoms. The term “cycloalkyl” refers to aliphatic cyclic alkyls having 3 to 10 carbon atoms in single or multiple cyclic rings, preferably 5 to 6 carbon atoms in a single cyclic ring. Non-limiting examples of suitable alkyl groups include methyl group, ethyl group, propyl group, isopropyl group, cyclopropyl group, butyl group, isobutyl group, sec-butyl group, tert-butyl group, cyclobutyl group, pentyl group, neo-pentyl group, cyclopentyl group, hexyl group, cyclohexyl group, 2-ethylhexyl, cyclohexylmethyl group, heptyl group, octyl group, nonyl group, decyl group, dodecyl group, tridecyl group, tetradcyl group, pentadecyl group, hexadecyl group, heptadecyl group, octadecyl group, nonadecyl group, cyclopentyl group, cyclohexyl group, and the like. Additional examples of cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcaryl, adamantyl, and spiro[4.5]decanyl groups, as well as their homologs, isomers, and the like. Alkyls can be substituted or unsubstituted. When the term is used with the “substituted” modifier, one or more hydrogen atoms has been independently replaced by any of the substituents disclosed herein.

As used herein, the term “heteroalkyl” refers to an alkyl as defined above having at least one carbon atom replaced by a heteroatom. Examples of cycloheteroalkyl groups include, among others, morpholinyl, thiomorpholinyl, pyranyl, imidazolidinyl, imidazolinyl, oxazolidinyl, pyrazolidinyl, pyrazolinyl, pyrrolidinyl, pyrrolinyl, tetrahydrofuranyl, tetrahydrothiophenyl, piperidinyl, piperazinyl, and the like. Heteroalkyls can be substituted or unsubstituted. When the term is used with the “substituted” modifier, one or more hydrogen atoms has been independently replaced by any of the substituents disclosed herein, or a substituent is bonded to a heteroatom, or both.

As used herein, the term “alkenyl” refers to a straight- or branched-chain hydrocarbon radical or moiety comprising only carbon and hydrogen atoms and having at least one carbon-carbon double bond, which can be internal or terminal Non-limiting examples of alkenyl groups include: —CH═CH₂ (vinyl), —CH═CH═CH₃, —CH═CHCH₂CH₃, —CH₂CH═CH₂(allyl), —CH₂CH═CHCH₃, —CH═CH—C₆H₅, —CH═CH—, CH(CH₃)CH₂—, and —CH═CHCH₂—. The groups, —CH═CHF, —CH═HCl, —CH═HBr, and the like. Examples of alkenyl groups include ethenyl, propenyl, butenyl, pentenyl, hexenyl, butadienyl, pentadienyl, hexadienyl groups, and the like. Alkenyls can be substituted or unsubstituted. When the term is used with the “substituted” modifier, one or more hydrogen atoms has been independently replaced by any of the substituents disclosed herein.

As used herein, the term “alkynyl” refers to a straight- or branched-chain hydrocarbon radical or moiety comprising only carbon and hydrogen atoms and having at least one carbon-carbon triple bond, which can be internal or terminal. The groups —C≡CH, —C≡CCH3, and —CH₂C≡CCH₃, are non-limiting examples of alkynyl groups. Examples of alkynyl groups include ethynyl, propynyl, butynyl, pentynyl, hexynyl, and the like. Alkynes can be substituted or unsubstituted. When the term is used with the “substituted” modifier, one or more hydrogen atoms has been independently replaced by any of the substituents disclosed herein.

As used herein, the term “carbonylalkyl” refers to a carbonyl group bonded, as a substituent, to an alkyl.

As used herein, the term “thioalkyl” and “alkylthio” refers to the group —S-alkyl.

As used herein, the term “hydoxyalkyl” refers to a hydroxyl group bonded, as a substituent, to an alkyl group.

As used herein, the term “alkylsilyl” refers to an alkyl group bonded, as a substituent, to a silyl group.

As used herein, the term “aminoalkyl” refers to an amino group bonded, as a substituent, to an alkyl.

As used herein, the term “aminoalkylthioalkyl” refers to an amino group bonded, as a substituent, to an alkyl group that is bonded, as a substituent, to a thioalkyl group.

As used herein, the term “aryl” refers to a monocyclic or polycyclic aromatic hydrocarbon radical or moiety comprising only carbon and hydrogen atom, wherein the carbon atoms form an aromatic ring structure. If more than one ring is present, the rings may be fused or not fused, or bridged. The term does not preclude the presence of one or more alkyl groups attached to the first aromatic ring or any additional aromatic ring present. The point of attachment can be through an aromatic carbon atom in the ring structure or a carbon atom of an alkyl group attached to the ring structure. Non-limiting examples of aryl groups include phenyl (Ph), toyl, xylyl, methylphenyl, (dimethyl)phenyl, —C6H4-CH2CH3 (ethylphenyl), naphthyl, and the monovalent group derived from biphenyl. Further examples of aryl groups having only aromatic carbocyclic ring(s) include phenyl, 1-naphthyl (bicyclic), 2-naphthyl (bicyclic), anthracenyl (tricyclic), phenanthrenyl (tricyclic), pentacenyl (pentacyclic), and like groups. Examples of polycyclic ring systems in which at least one aromatic carbocyclic ring is fused to one or more cycloalkyl and/or cycloheteroalkyl rings include, among others, benzo derivatives of cyclopentane (i.e., an indanyl group, which is a 5,6-bicyclic cycloalkyl/aromatic ring system), cyclohexane (i.e., a tetrahydronaphthyl group, which is a 6,6-bicyclic cycloalkyl/aromatic ring system), imidazoline (i.e., a benzimidazolinyl group, which is a 5,6-bicyclic cycloheteroalkyl/aromatic ring system), and pyran (i.e., a chromenyl group, which is a 6,6-bicyclic cycloheteroalkyl/aromatic ring system). When the term is used with the “substituted” modifier, one or more hydrogen atoms has been independently replaced by any of the substituents disclosed herein.

As used herein, the term “heteroaryl” refers to an aryl having at least one aromatic carbon atom in the ring structure replaced by a heteroatom. The term does not preclude the presence of one or more alkyl groups attached to the first aromatic ring or any additional aromatic ring present. The point of attachment can be through an aromatic carbon atom or aromatic heteroatom in the aromatic ring structure or a carbon atom of an alkyl group attached to the aromatic ring structure. Non-limiting examples of heteroaryl groups include furanyl, benzofuranyl, isobenzylfuranyl, imidazolyl, indolyl, isoindolyl, indazolyl, methylpyridyl, oxazolyl, pyridyl, pyrrolyl, pyrimidyl, pyrazinyl, quinolyl, quinazolyl, quinoxalinyl, thienyl, and triazinyl. Additional examples of heteroaryl groups include, for example, the 5- or 6-membered monocyclic and 5-6 bicyclic ring systems such as pyrrolyl, furyl, thienyl, pyridyl, pyrimidyl, pyridazinyl, pyrazinyl, triazolyl, tetrazolyl, pyrazolyl, imidazolyl, isothiazolyl, thiazolyl, thiadiazolyl, isoxazolyl, oxazolyl, oxadiazolyl, indolyl, isoindolyl, benzofuryl, benzothienyl, quinolyl, 2-methylquinolyl, isoquinolyl, quinoxalyl, quinazolyl, benzotriazolyl, benzimidazolyl, benzothiazolyl, benzisothiazolyl, benzisoxazolyl, benzoxadiazolyl, benzoxazolyl, cinnolinyl, IH-indazolyl, 2H-indazolyl, indolizinyl, isobenzofuyl, naphthyridinyl, phthalazinyl, pteridinyl, purinyl, oxazolopyridinyl, thiazolopyridinyl, imidazopyridinyl, furopyridinyl, thienopyridinyl, pyridopyrimidinyl, pyridopyrazinyl, pyridopyridazinyl, thienothiazolyl, thienoxazolyl, thienoimidazolyl groups, and the like. Further examples of heteroaryl groups include 4,5,6,7-tetrahydroindolyl, tetrahydroquinolinyl, benzothienopyridinyl, benzofuropyridinyl groups, and the like. Heteroaryls can be substituted or unsubstituted. When the term is used with the “substituted” modifier, one or more hydrogen atoms has been independently replaced by any of the substituents disclosed herein, or a substituent is bonded to a heteroatom, or both.

As used herein, the term “aralkyl” refers to an alkyl having at least one hydrogen atom replaced by an aryl or heteroaryl group. Non-limiting examples of aralkyls are: phenylmethyl (benzyl, Bn) and 2-phenyl-ethyl. The point of attachment can be through a carbon atom of the alkyl group or through an aromatic carbon atom or aromatic heteroatom in the aromatic ring structure of the aryl or heteroaryl group attached to the alkyl group. Aralkyls can be substituted or unsubstituted. When the term is used with the “substituted” modifier, one or more hydrogen atoms has been independently replaced by any of the substituents disclosed herein, or a substituent is bonded to a heteroatom, or both.

As used herein, the term “alkaryl” refers to an aryl or heteroaryl having at least one hydrogen atom replaced by an alkyl or heteroalkyl group. The point of attachment can be an aromatic carbon atom or aromatic heteroatom in the aromatic ring structure or a carbon atom of an alkyl group attached to the ring structure. Alkaryls can be substituted or unsubstituted. When the term is used with the “substituted” modifier, one or more hydrogen atoms has been independently replaced by any of the substituents disclosed herein, or a substituent is bonded to a heteroatom, or both.

As used herein, the term “heteroaralkyl” refers to a heteroaryl bonded, as a substituent, to an alkyl group.

As used herein, the term “heteroalkaryl” refers to a heteroalkyl bonded, as a substituent, to an aryl group.

As used herein, the terms “halide,” “halo,” and “halogen” refer to —F, —Cl, —Br, or —I.

As used herein, the term “substituent” and “substituted” refers to all permissible substituents of the compounds described herein. In the broadest sense, the permissible substituents include acyclic and cyclic, branched and unbranched, carbocyclic and heterocyclic, aromatic and nonaromatic substituents of organic compounds. Examples of substituents include, without limitation, nothing, alkyl, substituted alkyl, cycloalkyl, substituted cycloalkyl, heteroalkyl, substituted heteroalkyl, alkenyl, substituted alkenyl, alkynyl, substituted alkynyl, aryl, substituted aryl, heteroaryl, substituted heteroaryl, aralkyl, substituted aralkyl, alkaryl, substituted alkaryl, haloaryl, substituted haloaryl, alkoxy, substituted alkoxy, alkenyloxy, substituted alkenyloxy, alkynyloxy, substituted alkynyloxy, aryloxy, substituted aryloxy, aralkoxy, substituted aralkoxy, heteroaryloxy, substituted heteroaryloxy, acyloxy, substituted acyloxy, acyl, substituted acyl, halo (—F, —Cl, —Br, —I, etc.), hydrogen (—H), carboxyl (—COOH), hydroxy (—OH), oxo (O), hydroxyamino (—NHOH), nitro (—NO₂), cyano (—CN), isocyanate (—N═C═O), azido (—N₃), phosphate (e.g., —OP(O)(OH)₂, —OP(O)(OH)O—, deprotonated forms thereof, etc.), mercapto (—SH), thio (═S), thioether (═S—), sulfonamido (—NHS(O)₂—), sulfonyl (—S(O)₂—), sulfinyl (—S(O)₂—), amino, alkylamino, vinyl, allyl, any combinations thereof, and the like.

Additional examples of substituents include, but are not limited to, —NC, —S(R⁰)₂⁺, —N(R⁰)₃⁺, —SO₃H, —SO₂R⁰, —SO₃R⁰, —SO₂NHR⁰, —SO₂N(R⁰)₂, —COR⁰, —COOR⁰, —CONHR⁰,)CON(R⁰)₂, C₁₋₄₀ haloalkyl groups, C₆₋₁₄ aryl groups, and 5-14 membered electron-poor heteroaryl groups; where R⁰ is a C₁₋₂₀ alkyl group, a C₂₋₂₀ alkenyl group, a C₂₋₂₀ alkynyl group, a C₁₋₂₀ haloalkyl group, a C₁₋₂₀ alkoxy group, a C₆₋₁₄ aryl group, a C₃₋₁₄ cycloalkyl group, a 3-14 membered cycloheteroalkyl group, and a 5-14 membered heteroaryl group, each of which can be optionally substituted as described herein. Additional examples of substituents include, but are not limited to, —OR⁰, —NH₂, —NHR⁰, —N(R⁰)₂, and 5-14 membered electron-rich heteroaryl groups, where R⁰ is a C₁₋₂₀ alkyl group, a C₂₋₂₀ alkenyl group, a C₂₋₂₀ alkynyl group, a C₆₋₁₄ aryl group, or a C₃₋₁₄ cycloalkyl group.

As used herein, the term “percent transferred” refers to the % by mass of a species A transferred from a solution B to a solution C based on the total weight of species A in solution B prior to the transfer.

As used herein, “CB7” refers to cucurbit[7]uril; “OX” refers to ortho-xylene; “MX” refers to meta-xylene; “PX” refers to para-xylene; “OX@CB7” refers to a complex including ortho-xylene and cucurbit[7]uril; “MX@CB7” refers to a complex including meta-xylene and cucurbit[7]uril; and “PX-@CB7” refers to a complex including para-xylene and cucurbit[7]uril.

### Discussion

The present invention relates to the selective separation of one or more aromatic isomers from one or more isomers mixtures and, in particular, to the use of cucurbituril macrocycles, which are selective for one or more aromatic isomers, in liquid-liquid extraction systems and processes. As described herein, cucurbituril macrocycles may be included in aqueous solutions and used as liquid-liquid extraction solvents to separate some of the most challenging aromatic isomers mixtures in industry, including those in which the isomers have identical molecular weights, similar chemical structures, and close boiling points. The liquid-liquid extraction systems and processes disclosed herein can be performed under ambient temperature and ambient pressure, with substantially lower energy requirements than conventional separation processes. Furthermore, unlike conventional aromatic isomers separation processes, the liquid-liquid extraction systems and processes disclosed herein can separate aromatic isomers mixtures with high selectivity and/or high specificity, without the use of highly toxic and corrosive solvents.

While not wishing to be bound to a theory, it is believed that the separation of aromatic isomers from isomers mixtures can involve the formation of a host-guest complex in which the cucurbituril macrocycle is the host and the aromatic isomer to be extracted is the guest. For example, in some embodiments, the liquid-liquid extraction process can include a step in which an isomers mixture is contacted with an aqueous cucurbituril solution. Being selective for the extraction of at least one aromatic isomer, such as an ortho-substituted aromatic isomer or another select aromatic isomer, the cucurbituril macrocycle can be used to effectuate the transfer and/or extraction of the at least one aromatic isomer from the isomers mixture to the aqueous solution through the formation of the host-guest complex. It is believed that, upon contacting the isomers mixtures with the aqueous cucurbituril solution, the aromatic isomer to be extracted is taken up into and bound within the cavity of the cucurbituril macrocycle, where it resides, stabilized by various non-covalent interactions. As described below, the aromatic isomer can be released from the cavity of the cucurbituril macrocycle using an organic solvent.

The use of aqueous cucurbituril solutions as liquid-liquid extraction solvents offers numerous advantages over conventional liquid-liquid extraction solvents. For example, porous cucurbituril macrocycle, such as cucurbit[7]uril (CB7), may be used. In addition, most conventional porous materials are only selective for a specific benzene isomer due to their fixed pore sizes. However, the aqueous cucurbituril solutions disclosed herein, with their tunable properties and structures, can be used to separate a variety of aromatic isomers mixtures and thus have broader utility and range than conventional porous materials. For example, aqueous solutions of cucurbit[7]uril can be used to separate one or more of xylene isomers, dichlorobenzene isomers, dibromobenzene isomers, chlorotoluene isomers, and methyl-substituted benzene isomers, among other isomers, achieving more than 92% specificity after a single extraction cycle for certain separations. An additional advantage is that cucurbituril macrocycle exhibits higher chemical, moisture, and thermal stability than conventional porous materials, such as zeolites, metal-organic frameworks, porous coordination polymers, covalent organic frameworks, porous organic cages, and the like. The cucurbituril macrocycle can also be recovered and reused/recycled in one or more extraction cycles without any loss in performance

An example of a cucurbituril macrocycle that can used for the selective extraction of at least one aromatic isomer from at least one isomers mixture includes macrocycles with the following structural formula:

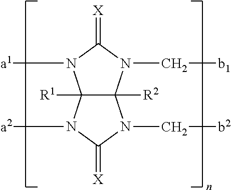

wherein:

a¹ indicates the point of attachment to b¹;

a² indicates the point of attachment to b²;

n is 1-20;

X is O, S, or NH; and

R¹ and R² are independently selected from the group consisting of hydrogen, H, optionally substituted C₁-C₃₀ alkyl group; optionally substituted C₂-C₃₀ alkenyl group; optionally substituted C₂-C₃₀ alkynyl group; optionally substituted C₂-C₃₀ carbonylalkyl group; optionally substituted C₁-C₃₀ thioalkyl group; optionally substituted C₁-C₃₀ alkylthiol group; optionally substituted C₁-C₃₀ hydroxyalkyl group; optionally substituted C₁-C₃₀ alkylsilyl group; optionally substituted C₁-C₃₀ aminoalkyl group; optionally substituted C₁-C₃₀ aminoalkylthioalkyl group; optionally substituted C₅-C₃₀ cycloalkyl group; optionally substituted C₂-C₃₀ heterocycloalkyl group; optionally substituted C₆-C₃₀ aryl group; optionally substituted C₆-C₃₀ arylalkyl group; optionally substituted C₄-C₃₀ heteroaryl group; and optionally substituted C₄-C₃₀ heteroarylalkyl group.

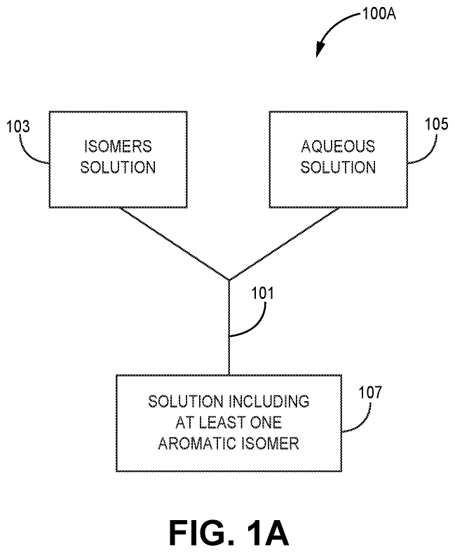

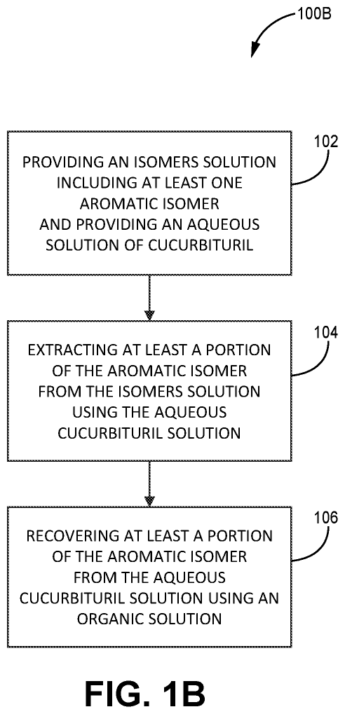

FIG. 1A is a flowchart of a liquid-liquid extraction process 100A for separating aromatic isomers, in accordance with one or more embodiments of the invention. The liquid-liquid extraction process of the present invention can utilize the cucurbituril macrocycle presented above. As shown in FIG. 1A, in some embodiments, the liquid-liquid extraction process 100A can comprise contacting 101 an isomers solution 103 including one or more aromatic isomers, with an aqueous solution 105 including a cucurbituril macrocycle, wherein the cucurbituril macrocycle is selective for the extraction of at least one of said aromatic isomers. The contacting 101 may be performed to obtain a solution 107 including at least one aromatic isomer from the isomers solution 103. In some embodiments, contacting 101 the isomers solution with the aqueous solution produces a first aqueous phase and a first organic phase, wherein the first aqueous phase includes the cucurbituril macrocycle and at least a portion of the aromatic isomer extracted from the isomers solution. In some embodiments, to recover the aromatic isomer from the first aqueous phase, the first aqueous phase is contacted with an organic solution to produce a second aqueous phase and a second organic phase, wherein the second organic phase includes at least a portion of the aromatic isomer from the first aqueous phase (and/or from the original isomers solution). In some embodiments, the second aqueous phase includes the cucurbituril macrocycle and is recycled and/or reused for use in one or more separation cycles (e.g., of a liquid-liquid extraction process).

Referring now to FIG. 1B, a flowchart of a liquid-liquid extraction process 100 for separating aromatic isomers is presented, in accordance with one or more embodiments of the present invention. As shown in FIG. 1B, the liquid-liquid extraction process 100 can comprise one or more of the following steps: providing 102 an isomers solution including at least one aromatic isomer and further providing an aqueous solution including a cucurbituril macrocycle; extracting 104 at least a portion of the aromatic isomer from the isomers solution, using the aqueous solution, to produce a first aqueous phase and a first organic phase, wherein the first aqueous phase includes at least a portion of the cucurbituril macrocycle from the aqueous solution and the portion of the aromatic isomer extracted from the isomers solution; recovering 106 at least a portion of the aromatic isomer from the first aqueous phase, using an organic solution, to produce a second aqueous phase and a second organic phase, wherein the second aqueous phase includes at least a portion of the cucurbituril macrocycle from the first aqueous phase and wherein the second organic phase includes the portion of the aromatic isomer recovered from the first aqueous phase; and recycling and/or reusing 108 the second aqueous phase for one or more extraction cycles (e.g., recycling/reusing in step 104).

Step 102 includes providing an isomers solution including at least one aromatic isomer and further providing an aqueous solution including a cucurbituril macrocycle.

The isomers solution can include one or more aromatic isomers, including at least one of the aromatic isomers to be extracted. The isomers solution can include one or more of mono-substituted aromatic isomers, di-substituted aromatic isomers, tri-substituted aromatic isomers, tetra-substituted aromatic isomers, penta-substituted aromatic isomers, hexa-substituted aromatic isomers, and so on. In some embodiments, the isomers include includes aromatic isomers which are at least di-substituted aromatic isomers. In some embodiments, the aromatic isomers, which are at least di-substituted aromatic isomers, include at least two substituents in one or more of an ortho position, a meta position, and a para position relative to each other. The isomers solution may include, for example, one or more of ortho-substituted aromatic isomers, para-substituted aromatic isomers, and meta-substituted aromatic isomers, each of which may independently have more two or more substituents attached to the aromatic compound forming the aromatic isomer. In another example, the isomers solution may include aromatic isomers including three or more substituents, such as for example C₃-benzenes, like trimethylbenzene isomers, among others.

In some embodiments, the isomers solution can include one or more benzene isomers. The benzene isomers can include one or more of an ortho-substituted benzene isomer, a para-substituted benzene isomer, and a meta-substituted benzene isomer. For example, in some embodiments, the isomers solution can include at least two benzene isomers. The at least two benzene isomers can include the ortho-substituted benzene isomer and at least one benzene isomer other than the ortho-substituted benzene isomer. Examples of benzene isomers other than the ortho-substituted benzene isomer include, without limitation, meta-substituted benzene isomers, para-substituted benzene isomers, and substituted benzene isomers other than ortho-substitute benzene isomers, meta-substituted benzene isomers, and para-substituted benzene isomers. In some embodiments, the isomers solution includes at least an ortho-substituted benzene isomer and a meta-substituted benzene isomer. In some embodiments, the isomers solution includes at least an ortho-substituted benzene isomer and a para-substituted benzene isomer. In some embodiments, the isomers solution includes at least an ortho-substituted benzene isomer and a benzene isomer other than an ortho-substituted benzene isomer, meta-substituted benzene isomer, and para-substituted benzene isomer. In some embodiments, the isomers solution includes at least an ortho-substituted benzene isomer and at least one of the following: a meta-substituted benzene isomer, a para-substituted benzene isomer, and a benzene isomer other than the ortho-substituted benzene isomer, meta-substituted benzene isomer, and para-substituted benzene isomer.

In some embodiments, the ortho-substituted benzene isomer has the chemical structure of formula (1A):

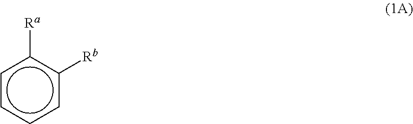

wherein R^{a} and R^{b} are independently halo, alkyl, alkoxy, hydroxyl, amino, alkylamino, cyano, isocyanate, nitro, vinyl, or allyl. In some embodiments, the ortho-substituted benzene isomer further includes from one to four additional substituents, wherein the substituents are defined above.

In some embodiments, the meta-substituted benzene isomer has the chemical structure of formula (1B):

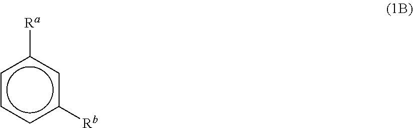

wherein R^{a} and R^{b} are as defined above. In some embodiments, the ortho-substituted benzene isomer further includes from one to four additional substituents, wherein the substituents are defined above.

In some embodiments, the para-substituted benzene isomer has the chemical structure of formula (1C):

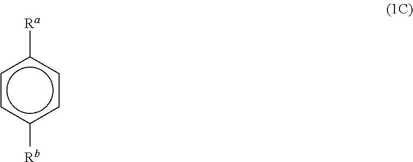

wherein R^{a} and R^{b} are as defined above. In some embodiments, the ortho-substituted benzene isomer further includes from one to four additional substituents, wherein the substituents are defined above.

In some embodiments, the isomers solution includes one or more of o-xylene, m-xylene, p-xylene, and ethylbenzene. In some embodiments, the isomers solution includes one or more of o-dibromobenzene, m-dibromobenzene and p-dibromobenzene. In some embodiments, the isomers solution includes one or more of o-dichlorobenzene, m-dichlorobenzene and p-dichlorobenzene. In some embodiments, the isomers solution includes one or more of o-bromotoluene, m-bromotoluene and p-bromotoluene. In some embodiments, the isomers solution includes one or more of o-chlorotoluene, m-chlorotoluene, and p-chlorotoluene. In some embodiments, the isomers solution includes one or more of 1-bromo-2-chlorobenzene, 1-bromo-3-chlorobenzene, and 1-bromo-4-chlorobenzene. In some embodiments, the isomers solution includes one or more of 2-chlorophenol, 3-chlorophenol, and 4-chlorophenol. In some embodiments, the isomers solution includes one or more of o-phenylenediamine, m-phenylenediamine, and p-phenylenediamine. In some embodiments, the isomers solution includes one or more of o-xylylenediamine, m-xylylenediamine, and p-xylylenediamine. In some embodiments, the isomers solution includes one or more of o-diethylbenzene, m-diethylbenzene, and p-diethylbenzene. In some embodiments, the isomer solution includes one or more of o-ethyltoluene, m-ethyltoluene, and p-ethyltoluene. In some embodiments, the isomer solution includes one or more of 1,2,3-trimethylbenzene (hemimellitene), 1,2,4-trimethylbenzene (pseudocumene), and 1,3,5-trimethylbenzene (mesitylene). In some embodiments, the isomers solution includes one or more of 1,2,4,5-tetramethylbenzene (durene), 1,2,3,5-tetramethylbenzene tetramethylbenzene (isodurene), and 1,2,3,4-tetramethylbenzene (prehnitene). In some embodiments, the isomers solution includes one or more of 1,4-diethylbenzene (para-diethylbenzene), 1,3-diethylbenzene (meta-diethylbenzene), and 1,2-diethylbenzene (ortho-diethylbenzene). In some embodiments, the isomers solution includes any combination of the foregoing.

The cucurbituril macrocycle can be dissolved or solubilized in water to obtain the aqueous solution of cucurbituril. Examples of water that can be used to form the aqueous solution of cucurbituril include, without limitation, tap water, distilled water, reverse osmosis water, neat water, and the like. The cucurbituril macrocycle included in the aqueous solution can be selective for the extraction of one or more aromatic isomers from the isomers solution. For examples, as further described below, in some embodiments, the cucurbituril macrocycle is selective for the extraction of an ortho-substituted aromatic isomer, such as an ortho-substituted benzene isomer. The ortho-substituted aromatic isomer and/or ortho-substituted benzene isomers may include any of those disclosed herein. The cucurbituril macrocycle can also not be selective for the extraction of aromatic isomers other than ortho-substituted aromatic isomers. In some embodiments, the cucurbituril macrocycle is not selective for the extraction of meta-substituted aromatic isomers. In some embodiments, the cucurbituril macrocycle is not selective for the extraction of para-substituted aromatic isomers. In some embodiments, the cucurbituril macrocycle is not selective for the extraction of substituted aromatic isomers, wherein said isomers are substituted benzene isomers other than ortho-substituted aromatic isomers, meta-substituted aromatic isomers, and/or para-substituted aromatic isomers.

The aqueous solution may include any of the cucurbituril macrocycles disclosed herein, including those having the structural formula presented above. In some embodiments, the aqueous solution includes cucurbit[7]uril. In some embodiments, the cucurbit[7]uril included in the aqueous solution has the chemical structure of formula (2):

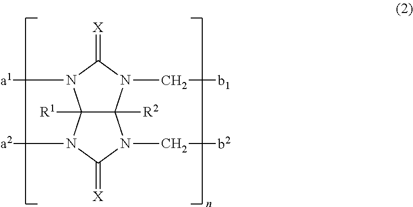

or hydrates, salts, and/or analogues thereof, wherein:

a¹ indicates the point of attachment to b¹;

a² indicates the point of attachment to b²;

n is 7;

X is O, S, or NH; and

R¹ and R² are independently selected from the group consisting of hydrogen, optionally substituted C₁-C₃₀ alkyl group; optionally substituted C₂-C₃₀ alkenyl group; optionally substituted C₂-C₃₀ alkynyl group; optionally substituted C₂-C₃₀ carbonylalkyl group; optionally substituted C₁-C₃₀ thioalkyl group; optionally substituted C₁-C₃₀ alkylthiol group; optionally substituted C₁-C₃₀ hydroxyalkyl group; optionally substituted C₁-C₃₀ alkylsilyl group; optionally substituted C₁-C₃₀ aminoalkyl group; optionally substituted C₁-C₃₀ aminoalkylthioalkyl group; optionally substituted C₅-C₃₀ cycloalkyl group; optionally substituted C₂-C₃₀ heterocycloalkyl group; optionally substituted C₆-C₃₀ aryl group; optionally substituted C₆-C₃₀ arylalkyl group; optionally substituted C₄-C₃₀ heteroaryl group; and optionally substituted C₄-C₃₀ heteroarylalkyl group.

In some embodiments, X is O. In some embodiments, X is S. In some embodiments, X is NH. IN some embodiments, R¹ and R² are H.

In some embodiments, the aqueous solution includes cucurbit[s,u]uril, wherein s+u=7. For example, in some embodiments, the aqueous solution includes cucurbit[1,6]uril. In some embodiments, the aqueous solution includes cucurbit[2,5]uril. In some embodiments, the aqueous solution includes cucurbit[3,4]uril. In some embodiments, the aqueous solution includes cucurbit[4,3]uril. In some embodiments, the aqueous solution includes cucurbit[5,2]uril. In some embodiments, the aqueous solution includes cucurbit[6,1]uril.

In some embodiments, the aqueous solution includes a cucurbituril macrocycle other than cucurbit[n]uril, wherein n is an integer selected from 1 to 6 and 8 to 20. For example, in some embodiments, the aqueous solution includes a cucurbituril macrocycle other than cucurbit[n]uril, where n is from 1 to 4. In some embodiments, the aqueous solution includes a cucurbituril macrocycle other than cucurbit[5]uril. In some embodiments, the aqueous solution includes a cucurbituril macrocycle other than cucurbit[6]uril. In some embodiments, the aqueous solution includes a cucurbituril macrocycle other than cucurbit[8]uril. In some embodiments, the aqueous solution includes a cucurbituril macrocycle other than cucurbit[n]uril, where n is from 9 to 12. In some embodiments, the aqueous solution includes a cucurbituril macrocycle other than cucurbit[n]uril, where n is from 13 to 15. In some embodiments, the aqueous solution includes a cucurbituril macrocycle other than cucurbit[n]uril, where n is from 16 to 18. In some embodiments, the aqueous solution includes a cucurbituril macrocycle other than cucurbit[n]uril, where n is from 19 to 20.

A solubilizing agent can optionally be added or included in the aqueous solution of cucurbituril. The solubilizing agent can be used to increase the solubility of the cucurbituril macrocycle in the aqueous solution. In some embodiments, the solubilizing agent includes a metal salt, such as NaCl or CsCl; an ammonium salt, such as NH₄Cl; an acid, such as a mineral or an organic acid (e.g., formic acid, citric acid, or trifluoroacetic acid (TFA)), and/or a polyhydroxylated organic compound, such as sugars (e.g., glucose, sucrose, or cyclodextrins), starch, or glycerol. Other suitable solubilizing agents for increasing the solubility of the cucurbituril macrocycle in aqueous solutions include, for example, coordination complexes, such as hexaamminecobalt (III) chloride.

In some embodiments, the cucurbituril macrocycle and aromatic isomer to be extracted (e.g., the ortho-substituted aromatic isomer) are provided in stoichiometric amounts. In some embodiments, the cucurbituril macrocycle is provided in stoichiometric excess of the aromatic isomer to be extracted (e.g., the ortho-substituted aromatic isomer).

Step 104 includes extracting at least a portion of the aromatic isomer from the isomers solution, using the aqueous solution of cucurbituril, to produce a first aqueous phase and a first organic phase. In this step, at least a portion of the aromatic isomer of interest is transferred from the isomers solution to the aqueous solution using a cucurbituril macrocycle, as the cucurbituril macrocycle is selective for the extraction of the said aromatic isomer of interest. In some embodiments, the first aqueous phase includes at least a portion of the cucurbituril macrocycle from the aqueous solution and the portion of the aromatic isomer extracted from the isomers solution.

While not wishing to be bound to a theory, it is believed that the mechanism by which at least a portion of the aromatic isomer is transferred from the isomers solution to the aqueous solution of cucurbituril can involve the formation of a host-guest complex, where the host includes the cucurbituril macrocycle and the guest includes the aromatic isomer. In some embodiments, the host-guest complex includes a cucurbituril macrocycle having a cavity and an aromatic isomer, wherein the aromatic isomer at least partially resides in the cavity of the curcurbituril. In some embodiments, the aromatic isomer can be bound within the cavity of the cucurbituril macrocycle. In some embodiments, the host-guest complex is stabilized by non-covalent interactions. In some embodiments, the cucurbituril macrocycle is selective for the extraction of the ortho-substituted aromatic isomer to the extent that no host-guest complexes are formed (at least to any appreciable degree) in which the guest species is an aromatic isomer other than the ortho-substituted aromatic isomer. In some embodiments, the cucurbituril macrocycle is selective for the extraction of aromatic isomers having other substitution patterns, such as for example 1,2,3-trimethylbenzene.

The first aqueous phase generally includes the aqueous solution of cucurbituril, as well as the portion of the aromatic isomer transferred from the isomers solution thereto. In some embodiments, the first aqueous phase includes a host-guest complex in which the cucurbituril macrocycle is the host species and the aromatic isomer to be extracted is the guest species. In some embodiments, the first aqueous phase is substantially free of host-guest complexes in which the cucurbituril macrocycle is the host species and an aromatic isomer other than the ortho-substituted aromatic isomer is the guest species. In some embodiments, the first aqueous phase is substantially free of aromatic isomers other than ortho-substituted aromatic isomers. For example, in some embodiments, the first aqueous phase is substantially free of meta-substituted aromatic isomers, para-substituted aromatic isomers, and aromatic isomers other than ortho-substituted aromatic isomers, meta-substituted aromatic isomers, and para-substituted aromatic isomers.

The cucurbituril macrocycle may be selective for the extraction of ortho-substituted aromatic isomers and other select aromatic isomers, such as those with different substitution patterns and/or those which are multi-substituted (e.g., have three or more substituents attached thereto). In some embodiments, the cucurbituruil macrocycle selective extracts one or more of the following: one or more of o-xylene, o-dibromobenzene, o-dichlorobenzene, o-bromotoluene, o-chlorotoluene, 1-bromo-2-chlorobenzene, 2-chlorophenol, o-phenylenediamine, o-xylylenediamine, o-diethylbenzene, 1,2,3-trimethylbenzene, 1,2-diethylbenzene (ortho-diethylbenzene), 1,2,4,5-tetramethylbenzene (durene), 1,2,3,5-tetramethylbenzene tetramethylbenzene (isodurene), and 1,2,3,4-tetramethylbenzene (prehnitene). The first aqueous phase thus may include one or more of the foregoing aromatic isomers. In other embodiments, other isomers of the foregoing may be selectively extracted by the cucurbituril macrocycle.

The first organic phase generally includes the isomers solution less the portion of the aromatic isomer (e.g., ortho-substituted aromatic isomer) transferred to the aqueous solution of cucurbituril and/or first aqueous phase. The first organic phase can include aromatic isomers not transferred from the isomers solution to the aqueous solution of cucurbituril via the extracting. For example, in some embodiments, the first organic phase includes at least one of the following: meta-substituted aromatic isomers, para-substituted aromatic isomers, and aromatic isomers other than ortho-substituted aromatic isomers, meta-substituted aromatic isomers, and para-substituted aromatic isomers. In some embodiments, the first organic phase includes the portion of ortho-substituted aromatic isomers not transferred to the aqueous solution of cucurbituril (if any). In some embodiments, the first organic phase is substantially free of ortho-substituted aromatic isomers and/or of 1,2,3-trimethylbenzene.

The extracting can be performed by contacting the isomers solution and aqueous solution. The contacting can proceed simultaneously or substantially simultaneously, or the contacting can proceed sequentially in any order. In some embodiments, the extracting can include admixing the isomers solution and aqueous solution. In some embodiments, the extracting can include agitating the isomers solution and aqueous solution. In some embodiments, the extracting can include mechanically mixing the isomers solution and aqueous solution. In some embodiments, the extracting can include combining the isomers solution and aqueous solution in a first extraction unit. In some embodiments, the extracting can include feeding the isomers solution and aqueous solution to a first extraction unit. In some embodiments, the extracting can include adding the isomers solution and aqueous solution to the first extraction unit.

The conditions and duration of the extracting step should be sufficient to transfer at least a portion of the aromatic isomer to be extracted (e.g., the ortho-substituted aromatic isomer) from the isomers solution to the aqueous solution of cucurbituril. In some embodiments, the extracting step proceeds under ambient conditions (e.g., ambient temperatures and pressures) to reduce or minimize the energy requirements of, and the energy consumed by, the liquid-liquid extraction process. For example, in some embodiments, the isomers solution and aqueous extractant are contacted at ambient temperatures. In some embodiments, ambient temperatures include temperatures of about 25° C.±5° C. In some embodiments, the isomers solution and aqueous extractant are contacted at ambient pressures. In some embodiments, ambient pressures include atmospheric pressures. In some embodiments, the contacting duration can range from a few seconds to weeks. In some embodiments, the duration of the contacting is from 5-10 minutes. In some embodiments, the duration of the contacting is about 10-15 minutes. In some embodiments, the duration of the contacting is about 15-20 minutes. In some embodiments, the duration of the contacting is greater than 20 minutes. In some embodiments, the contacting of the isomers solution and aqueous extractant includes or is followed by a settling period of any duration.

In some embodiments, the percent transferred of the aromatic isomer to be extracted (e.g., the ortho-substituted aromatic isomer) from the isomers solution to the aqueous solution is up to about 99%, or any incremental value or subrange between 0% and 99%. In some embodiments, the percent transferred is up to about 97%. In some embodiments, the percent transferred is up to about 96.5%. In some embodiments, the percent transferred is up to about 96%. In some embodiments, the percent transferred is up to about 95.5%. In some embodiments, the percent transferred is up to about 95%. In some embodiments, the percent transferred is up to about 94.5%. In some embodiments, the percent transferred is up to about 94%. In some embodiments, the percent transferred is up to about 93.5%. In some embodiments, the percent transferred is up to about 93%. In some embodiments, the percent transferred is up to about 92.5%. In some embodiments, the percent transferred is up to about 92%. In some embodiments, the percent transferred is up to about 91.5%. In some embodiments, the percent transferred is up to about 91%. In some embodiments, the percent transferred is up to about 90.5%. In some embodiments, the percent transferred is up to about 90%.

In some embodiments, the percent transferred of the cucurbituril macrocycle from the aqueous solution to the isomers solution is less than 0.5%. In some embodiments, the percent transferred of the cucurbituril macrocycle from the aqueous solution to the isomers solution is less than 1%. In some embodiments, the percent transferred of the cucurbituril macrocycle from the aqueous solution to the isomers solution is less than 2%. In some embodiments, the percent transferred of the cucurbituril macrocycle from the aqueous solution to the isomers solution is less than 3%. In some embodiments, the percent transferred of the cucurbituril macrocycle from the aqueous solution to the isomers solution is less than 4%. In some embodiments, the percent transferred of the cucurbituril macrocycle from the aqueous solution to the isomers solution is less than 5%. In some embodiments, the percent transferred of the cucurbituril macrocycle from the aqueous solution to the isomers solution is less than 20%, or any incremental value or subrange between 0% and 20%.

Step 106 includes recovering at least a portion of the extracted aromatic isomer (e.g., the ortho-substituted aromatic isomer, etc.) from the first aqueous phase, using an organic solution, to produce a second aqueous phase and a second organic phase. In this step, at least a portion of the extracted aromatic isomer is transferred from the first aqueous phase to the organic solution using an organic solvent which is selective for the extraction of the extracted aromatic isomer (e.g., the ortho-substituted aromatic isomer, etc.), thereby producing an organic solution (e.g., as the second organic phase) in which the extracted aromatic isomer (e.g., the ortho-substituted aromatic isomer, etc.) is the majority component and regenerating the aqueous cucurbituril (e.g., as the second aqueous phase) which can be recycled and reused in an additional one or more extraction/separation cycles.

The organic solution can be selected to break or at least weaken the non-covalent interactions stabilizing or binding the extracted aromatic isomer (e.g., the ortho-substituted aromatic isomer, etc.) within the cavity of the cucurbituril macrocycle (e.g., so as to release said aromatic isomer from the cavity of the cucurbituril macrocycle). The organic solution can include a single organic solvent or the organic solution can include a mixture of organic solvents. In some embodiments, the organic solution can include one or more of hexane, pentane, cyclohexane, heptane, carbon tetrachloride, chloroform, 1,2-dichloroethane, dichloromethane, diethyl ether, dimethyl formamide, ethyl acetate, or methyl tert-butyl ether. In some embodiments, the organic solvent is hexane, pentane, cyclohexane, or a mixture thereof. In some embodiments, the organic solvent is carbon tetrachloride, chloroform, 1,2-dichloroethane, dichloromethane, or a mixture thereof. In some embodiments, the organic solvent is hexane, pentane, cyclohexane, heptane, carbon tetrachloride, chloroform, 1,2-dichloroethane, dichloromethane, or a mixture thereof. In some embodiments, the organic solvent is hexane, pentane, cyclohexane, heptane, carbon tetrachloride, chloroform, 1,2-dichloroethane, dichloromethane, diethyl ether, dimethyl formamide, ethyl acetate, methyl tert-butyl ether, or a mixture thereof.

The second organic phase can include the organic solution and the portion of the extracted aromatic isomer (e.g., the ortho-substituted aromatic isomer, etc.) transferred from the first aqueous phase. In some embodiments, the second organic phase includes the organic solution and an ortho-substituted aromatic isomer, and further includes one or more aromatic isomers other than the ortho-substituted aromatic isomer. For example, in some embodiments, the second organic phase includes the organic solution and ortho-substituted aromatic isomer and further includes at least one of the following aromatic isomers: ortho-substituted aromatic isomers, meta-substituted aromatic isomers, para-substituted aromatic isomers, and aromatic isomers other than ortho-substituted aromatic isomers, meta-substituted aromatic isomers, para-substituted aromatic isomers. In some embodiments, the second organic phase is substantially free of aromatic isomers other than the ortho-substituted aromatic isomer. For example, in some embodiments, the second organic phase is substantially free of at least one of the following aromatic isomers: ortho-substituted aromatic isomers, meta-substituted aromatic isomers, para-substituted aromatic isomers, and aromatic isomers other than ortho-substituted aromatic isomers, meta-substituted aromatic isomers, para-substituted aromatic isomers. In some embodiments, the aromatic isomers are benzene isomers. In some embodiments, the second organic phase includes 1,2,3-trimethylbenzene and is optionally substantially free of 1,2,4-trimethylbenzene and/or 1,3,5-trimethylbenzene.

Gas chromatograph measurements, among other measurements, can be carried out to determine the relative amount of aromatic isomers in the second organic phase. For example, in some embodiments, the aromatic isomers include at least about 90% of the ortho-substituted aromatic isomer, at least about 91% of ortho-substituted aromatic isomer, at least about 92% of ortho-substituted aromatic isomer, at least about 93% of ortho-substituted aromatic isomer, at least about 94% of ortho-substituted aromatic isomer, at least about 95% of ortho-substituted aromatic isomer, at least about 96% of ortho-substituted aromatic isomer, at least about 97% of ortho-substituted aromatic isomer, at least about 98% of ortho-substituted aromatic isomer, at least about 99% of ortho-substituted aromatic isomer, with the balance including aromatic isomers other than the ortho-substituted aromatic isomer. In other embodiments, the aromatic isomers include from 25% to 100% of ortho-substituted aromatic isomer, or any incremental value or subrange between that range. In some embodiments, the ortho-substituted aromatic isomer is the majority isomer, wherein the aromatic isomers include greater than about 50% of ortho-substituted aromatic isomer.

The second aqueous phase can include the aqueous solution of cucurbituril less the portion of the ortho-substituted aromatic isomer transferred to the organic solution and/or second organic phase. In some embodiments, the second aqueous phase is substantially free of the ortho-substituted aromatic isomer and/or the extracted aromatic isomer, thus regenerating the aqueous solution of cucurbituril which can be recycled and/or reused in additional extraction cycles. In some embodiments, the second aqueous phase is substantially free of at least one of the following aromatic isomers: ortho-substituted aromatic isomers, meta-substituted aromatic isomers, para-substituted aromatic isomers, and aromatic isomers other than ortho-substituted aromatic isomers, meta-substituted aromatic isomers, para-substituted aromatic isomers. In other embodiments, the second aqueous phase can include trace amounts or less than 5% by weight or volume of aromatic isomers not transferred to the organic solution and/or second organic phase.

The recovering can be performed by extracting the extracted aromatic isomer into the organic solution from the first aqueous phase. For example, in some embodiments, the recovering can be performed by contacting the first aqueous phase and the organic solution. The contacting can proceed simultaneously (e.g., substantially simultaneously) or sequentially in any order. In some embodiments, the recovering can include admixing the first aqueous phase and the organic solution. In some embodiments, the recovering can include agitating the first aqueous phase and the organic solution. In some embodiments, the recovering can include mechanically mixing the first aqueous phase and the organic solution. In some embodiments, the recovering can include combining the first aqueous phase and the organic solution in a second extraction unit. In some embodiments, the recovering can include feeding the first aqueous phase and the organic solution to a second extraction unit. In some embodiments, the recovering can include adding the first aqueous phase and the organic solution to the second extraction unit. In some embodiments, prior to performing the recovering step, the first aqueous phase and the first organic phase can be separated by centrifugation, filtration, or other similar processes.

Step 108 includes recycling the second aqueous phase produced in step 106 to step 104. The aqueous solution of cucurbituril can be regenerated in step 106 through decomplexation of the cucurbituril macrocycle and ortho-substituted aromatic isomer and transfer of the ortho-substituted aromatic isomer from the first aqueous phase to the organic solution and/or second organic phase. The second aqueous phase can include the regenerated aqueous solution of cucurbituril which can be recycled to step 104 and reused in one or more extraction cycles. In some embodiments, prior to performing the recycling step, the second aqueous phase and the second organic phase can be separated by centrifugation, filtration, or other similar processes.

In some embodiments, the liquid-liquid extraction process for separating aromatic isomers can include a single step in which the aqueous solution of cucurbituril is contacted with an ortho-substituted aromatic isomer, wherein the cucurbituril macrocycle and ortho-substituted aromatic isomer form a host-guest complex in which the ortho-substituted aromatic isomer at least partially resides in the cavity of the cucurbituril macrocycle.

While ortho-substituted aromatic isomers are described herein as forming host-guest complexes with the cucurbituril macrocycle, the cucurbituril macrocycle can be selective for aromatic isomers other than ortho-substituted aromatic isomers. For example, in some embodiments, the cucurbituril macrocycle is selective for para-substituted aromatic isomers. In some embodiments, the cucurbituril macrocycle is selective for meta-substituted aromatic isomers. The selectivity of the cucurbituril macrocycle can be dependent on a variety of different factors, such as thermodynamics, complexation kinetics, dimensional aspects, and so on. For example, factors such as complexation binding constants (Kₐ), decomplexation rate constants (k₁), aspect ratios based on dimensions (a/b), and the like can be modified and/or adjusted to modulate the selectivity of the cucurbituril macrocycle for aromatic isomers other than ortho-substituted aromatic isomers.

The liquid-liquid extraction process is general and thus the implementation of the liquid-liquid extraction process is not particularly limited. The liquid-liquid extraction process can be implemented using various extraction systems. For example, the liquid-liquid extraction processes can be implemented using extraction towers, mixer-settlers, single-stage unit operations, and/or multi-stage unit operations, such as countercurrent multi-stage cascades. The manner in which the liquid-liquid extraction process is carried out is also not particularly limited. For example, in some embodiments, the liquid-liquid extraction process is implemented in a system operating as a continuous process. In some embodiments, the liquid-liquid extraction process is implemented in a system operating as a batch process. In some embodiments, the liquid-liquid extraction process is implemented in a system operating as a semi-continuous and/or semi-batch process in which at least one unit operation of said system is operated as a continuous process and/or at least one unit operation of said system is operated as a batch process.

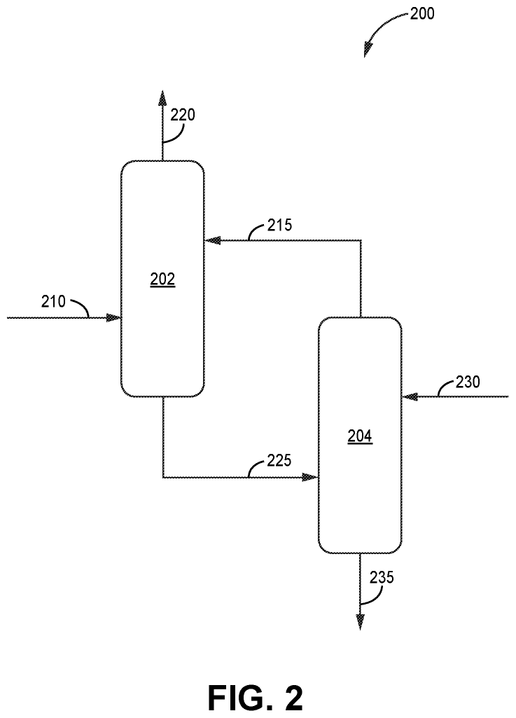

One example of an implementation of the liquid-liquid extraction process is shown in FIG. 2, which is a schematic diagram of a liquid-liquid extraction system 200 for separating aromatic isomers, in accordance with one or more embodiments of the present invention. As shown in FIG. 2, the liquid-liquid extraction system can comprise a first extraction tower 202 and a second extraction tower 204. In the illustrated embodiment, the second extraction tower 204 is provided downstream of the first extraction tower 202. Although not shown, the liquid-liquid extraction system 200 can optionally further comprise other auxiliary components (not shown) including, for example and without limitation, conduits, pipes, diverters, pumps, control systems, sensors, valves, and the like.

The first extraction tower 202 can be in fluid communication with inlet 210, inlet 215, outlet 220, and outlet 225. In some embodiments, the isomers solution can enter the first extraction tower 202 through inlet 210. In some embodiments, the aqueous solution of cucurbituril (and/or second aqueous phase from the first extraction tower 204) can enter the first extraction tower 202 through inlet 215. In some embodiments, the first organic phase can exit the first extraction tower 202 through outlet 220. In some embodiments, the outlet 220 can be in fluid communication with inlet 210 and the first organic phase can be recycled to the first extraction tower 202 for one or more extraction cycles. In some embodiments, the first aqueous phase can exit the first extraction tower 202 through outlet 225 and, from the first extraction tower, the first aqueous phase can be directed to the second extraction tower 204 via inlet 225 which can be the same as outlet 225.

The second extraction towner can be in fluid communication with inlet 225, inlet 230, outlet 215, and outlet 235. In some embodiments, inlet 225 is the same as outlet 225 and the first aqueous phase from the first extraction tower 202 can enter the second extraction tower 204 through inlet 225. In some embodiments, the organic solution enters the second extraction tower 204 through inlet 230. In some embodiments, the second aqueous phase can exit the second extraction tower 204 through outlet 215 and, from the second extraction tower, the second aqueous phase can be directed to the first extraction tower 204 via inlet 215 which can be the same as outlet 215. In some embodiments, the second organic phase can exit the second extraction tower 204 through outlet 235 and, from the second extraction towner 204, the second organic phase can be directed to other unit operations (not shown) for further processing and/or storage. For example, in some embodiments, further processing of the second organic phase includes isolating or separating the ortho-substituted aromatic isomer from the organic solvent via evaporation, distillation, and other separation processes.

In one embodiment, the liquid-liquid extraction towner 200 includes a first extraction tower 202, a second extraction tower 204 downstream from the first extraction towner 202, and optionally one or more of the auxiliary components discussed above. The first extraction tower 202 can be in fluid communication with isomer feed stream 210, cucurbituril recycle stream 215, organic raffinate stream 220, and aqueous extract stream 225. The second extraction tower 204 can be in fluid communication with aqueous extract stream 225, organic solvent stream 230, organic raffinate 235, and cucurbituril recycle stream 215. The isomer feed stream 210 can include the isomers solution. The cucurbituril recycle stream 215 can include the aqueous solution of cucurbituril and/or the second aqueous phase. The organic raffinate stream 220 can include the first organic phase. The aqueous extract stream 225 can include the first aqueous phase. The organic solvent stream 230 can include the organic solution. The organic raffinate 235 can include the second organic phase. The cucurbituril recycle stream 215 can include the second aqueous phase.

In another embodiment, the liquid-liquid extraction system can include a first extraction tower in fluid communication with an isomer feed stream and an aqueous cucurbituril feed stream, wherein the isomer feed stream includes an ortho-substituted aromatic isomer and at least one aromatic isomer other than the ortho-substituted aromatic isomer and wherein aqueous cucurbituril feed stream includes a cucurbituril macrocycle which is selective for the extraction of the ortho-substituted aromatic isomer; and a second extraction tower downstream from the first extraction tower, the second extraction tower in fluid communication with an aqueous extract stream from the first extraction tower and an organic solvent stream, wherein the aqueous extract stream includes the ortho-substituted aromatic isomer and cucurbituril macrocycle and wherein the organic solvent stream includes an organic solvent which is selective for the extraction of the ortho-substituted aromatic isomer.

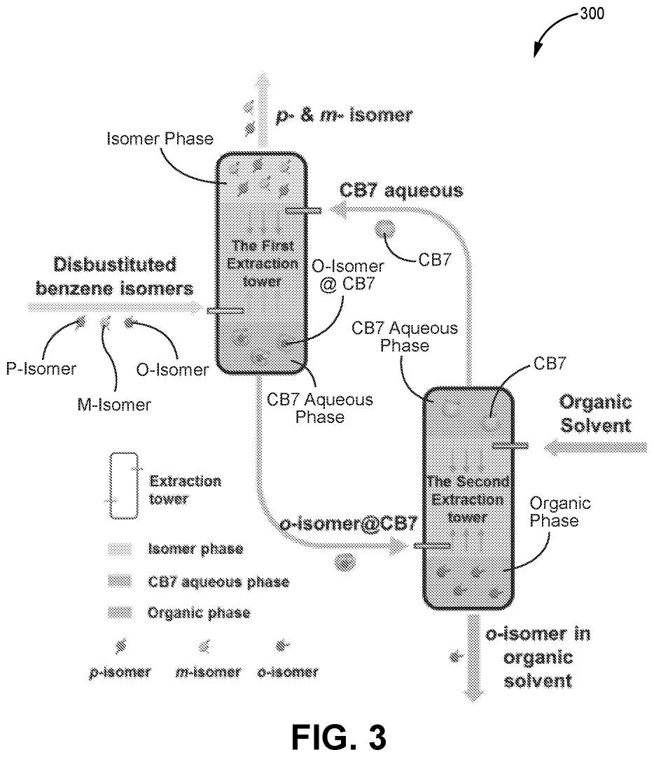

FIG. 3 is a schematic diagram of a liquid-liquid extraction system and process 300, in accordance with one or more embodiments of the present invention. In accordance with the process, CB7 was dissolved in neat water to form a CB7 aqueous solution (4 mg/ml). Then substituted benzene isomers (1:1:1 mixtures) were added onto the CB7 aqueous solution in the first extraction tower. After stirring, the un-emulsified water phase was separated from the xylene phase and transferred to the second extraction tower. In the second extraction tower, the water phase was extracted using an organic solvent (e.g., hexane, pentane, cyclohexane, etc.). For nuclear magnetic resonance (NMR) experiments, CDCl₃ was used as the organic solvent for extraction. After extraction, the organic phase was separated from water phase, and the CB7 aqueous solution was reused. The NMR and Gas chromatograph (GC) analysis showed the separation efficiency was 92.2% for ortho-xylene, 96.9% for ortho-dichlorobenzene, 96.7% for dibromobenzene, 96.8% for chlorotoluene in one extraction cycle. The aqueous CB7 solution was able to separate these compounds without any loss in performance after recycling 5 times.

In some embodiments, a liquid-liquid extraction method is provided for separating and purifying various ortho-substituted benzene isomers using cucurbit[7]uril (CB7) aqueous solution. In some embodiments, the separation process achieves more than 92% specificity after one extraction cycle at ambient temperature and pressure. Thermodynamic and kinetic analysis demonstrates that the ortho-substituted isomer can exhibit a stronger binding ability and slower decomplexation constant rate than the para- and meta-substituted isomers when hosted by CB7. Optimized host-guest models simulated by density-functional theory (DFT) calculation indicate that ortho-substituted isomer with the smallest aspect ratio is a good match for the spherical interior cavity of CB7, resulting in highly stable complexes. The method proceeded by a shape-induced separation based on host-guest chemistry with no energy costs, thereby improving the quality and lowering the costs of critical industrial separations.

The following Examples are intended to illustrate the above invention and should not be construed as to narrow its scope.

### EXAMPLES

In the following Examples, an aqueous solution CB7 was employed as a liquid-liquid extraction solvent and used to separate four groups of ortho-substituted benzene isomers from their corresponding 1:1:1 mixture under ambient conditions. The separation efficiency exceeded 92% in one extraction cycle. Xylene isomers were selected as model molecules to investigate the complexation behavior with CB7 in the aqueous phase. ¹H NMR spectra and 2D NOESY spectra of the host-guest experiments showed that all three xylenes could enter the cavity of CB7 to form host-guest complexes. The binding constant (Kₐ) for the complexes between CB7 and xylene isomers were obtained from isothermal titration calorimetry (ITC) experiments and indicated that ortho-xylene (OX) was bound more strongly to CB7, which was 21 times greater than para-xylene (PX) and 27 times greater than meta-xylene (MX). Kinetic analysis from the variable-temperature ¹H NMR experiments demonstrated that even though the three xylene guests had similar complexation rate constants, k₁, OX had the slowest decomplexation rate constant k₋₁. Structure analysis of PX, MX and OX showed that OX possessed the smallest aspect ratio (e.g., the molecule length divided by molecule width), which suggested a better match with the spherical cavity of CB7. Theoretical calculations supported the proposed shape fitting effect between xylene isomers and CB7. In water, OX tended to form parallel inclusion with CB7 which showed a lower binding energy than the vertical complexes PX@CB7 and MX@CB7. Moreover, aqueous CB7 showed an excellent separation efficiency (about 96%) of other disubstituted benzene isomers such as dichlorobenzenes (DCB), dibromobenzenes (DBB), and chlorotoluenes (CT).

The examples herein demonstrate that the macrocycle host CB7 can easily separate four kinds of ortho-substituted benzene compounds (OX, O-DCB, O-DBB, O-CT) from their corresponding isomers by a liquid-liquid extraction process. Thermodynamic and kinetic experiments performed by using xylene molecules as model guests showed that OX had the highest binding constant with CB7 which was 21 times higher than MX and 27 times higher than PX. DFT optimized structures showed that OX had a different binding mode with CB7 due to the discoid molecular shape. It is believed that the spherical cavity of CB7 matches the discoid shape of ortho-substituted benzene isomers, leading to the formation of highly stable complexes and thus a selective shape-induced separation based on host-guest chemistry with no energy costs. The liquid-liquid extraction method reported here has at least the following advantages: (i) The porous material CB7 used is commercially available; (ii) CB7 has high chemical, moisture, and thermal stability compared to most of the reported porous materials, such as zeolites, MOFs, PCPs, COFs, or POCs. (iii) The whole separation process is performed under ambient temperature and pressure which means easy operation and low energy consumption; and (iv) Most porous materials only show high selectivity for a specific benzene isomer due to the fixed pore size, whereas CB7 can separate four kinds of benzene isomers with more than 92% specificity after one extraction cycle.

### Example 1

### Materials and Methods

All reagents including CB7 hydrate were purchased from Alfa Aesar and Sigma Aldrich, and used as supplied without further purification. Purity of the CB7 hydrate was checked by using ¹H-NMR spectroscopy and UV-vis titration with cobaltocenium hexafluorophosphate which resulted in the sample containing 71% CB7.

NMR spectra were recorded using 600 MHz, 700 MHz and 950 MHz BRUKER AVANCE III spectrometers. Chemical shifts were reported in ppm relative to the residual internal non-deuterated solvent signals (D₂O: δ=4.79 ppm, CDCl₃: δ=7.26 ppm). Decomplexation rates were calculated by using Topspin 3.5 with DNMR (Dynamic NMR) line shape analysis module which is based on the average density matrix theory. Kinetic parameters of the complexes between CB7 and xylenes were estimated using the Eyring equation, where R is the ideal gas constant, h is Plank's constant and kB is Boltzmann's constant.

ΔG^{≠}=−RTln(kh/k_{B}T)

ln(k/T)=—(ΔH^{≠}/RT)+(ΔS^{≠}/R)+ln(k_{B}/h)

Isothermal titration experiments (ITC) were carried out using Nano-ITC (TA instruments) at 25° C. in neat water. All solutions were degassed prior to titration. The concentration of the xylene solutions was determined by absorbance spectroscopy after degassing. In a typical experiment, a 5 μL CB7 solution (the first injection was 2 μL) with 250 seconds spacing was injected 41 times into the ITC cell, which contained the xylene solution. Heats of dilution were determined by titration of the CB7 into water. The first data point was always removed from the data set prior to curve fitting. The data were analyzed using NanoAnalyze software (TA instruments) after deduction of the enthalpy of dilution from the total enthalpy change. The knowledge of the complex stability constant (Kₐ) and molar reaction enthalpy (ΔH) enabled the calculation of the standard free energy (ΔG) and entropy changes (ΔS).

ΔG=−RTlnKₐ=ΔH−TΔS.

DFT calculations were performed using the Gaussian 09 software package. Geometry optimization and frequency analysis of the CB7 complexes with xylenes were performed at ωB97XD/6-31G(d) level and then high accuracy energies were calculated at ωB97XD/6-311+G(d) level. To simulate binding energies water phase, single point energies were calculated at ωB97XD/6-311+G(d) level in polarized continuum model (PCM) with water as the solvent. The molecular volumes of the isomers were first optimized by a semiempirical method (AM1) and then calculated according to the van der Waals volume model included in the Hyperchem software package. The van der Waals radius for the molecular size used the following values: C=1.70 Å, O=1.60 Å, N=1.65 Å, H=1.20 Å, Br=1.85 Å, Cl=1.75 Å and F=1.47 Å.

GC measurements were carried out using an Agilent 7890A instrument configured with an FID detector and a HP-INNOWAX column (60 m×0.320 mm×0.5 μm). The following GC method was used. The oven was programmed in a definite temperature (65° C. for CFB isomers, 75° C. for xylene isomers, 95° C. for CT isomers, 125° C. for DCB isomers and 145° C. for DBB isomers) with a 30 min hold; injection temperature was 180° C.; detector temperature was 250° C. with hydrogen, air, and make-up flow-rates of 40, 400, and 15 mL min⁻¹, respectively; helium (carrier gas) flowrate was 3.0 mL min⁻¹. The samples were injected in the split mode (1:1).

### Example 2

### Separation Process

For the separation process, 20 mg of CB7 hydrate was dissolved in 5 ml of neat water. Then 5 ml of xylene isomers (1:1:1 mixture of OX: MX: PX) was added into the above solution. After stirring for 10 minutes, the water phase was separated from the xylene phase. The emulsifying xylene liquid in water phase could be removed by centrifugation or filtration. The unemulsified water phase was then extracted by organic solvent (hexane, pentane, cyclohexane, and/or other water in-mixable organic solvents). After stirring for 10 minutes, the aqueous solution of CB7 was separated from the organic phase and reused again in the first extraction setup.

### Example 3

### Xylene Isomers Separation Process

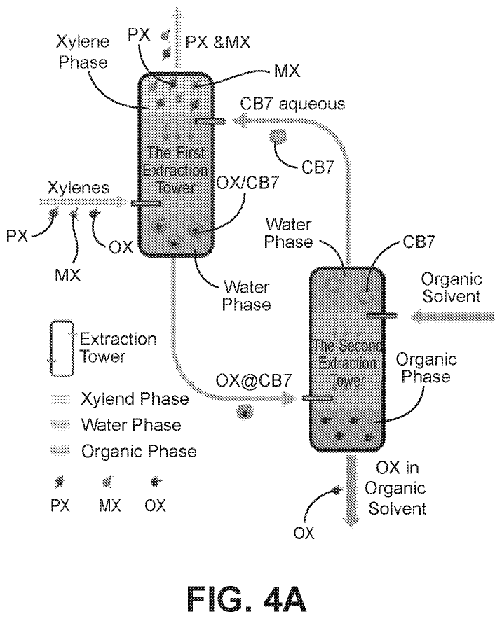

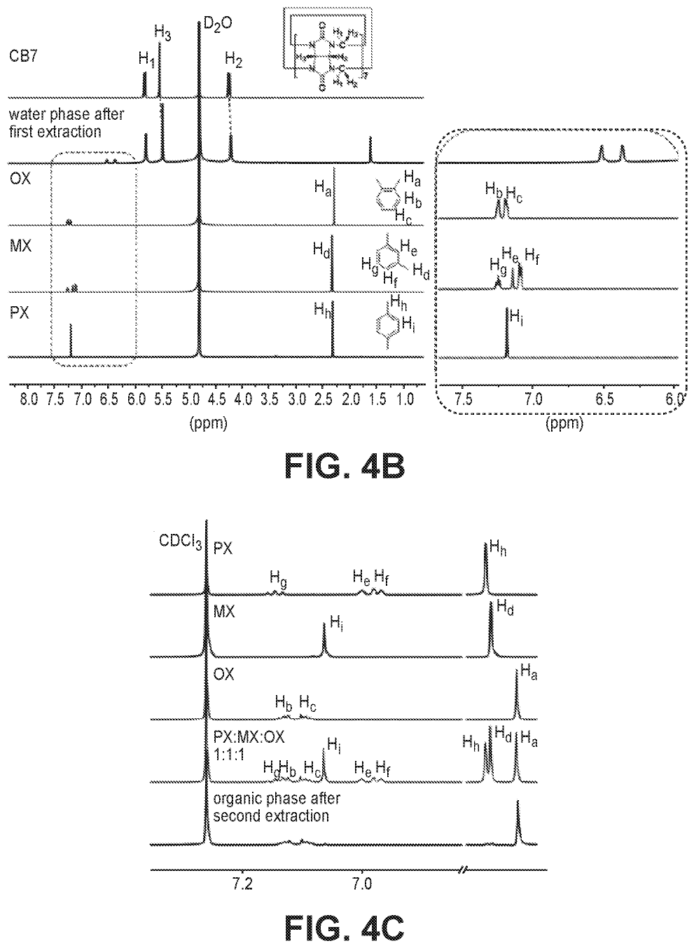

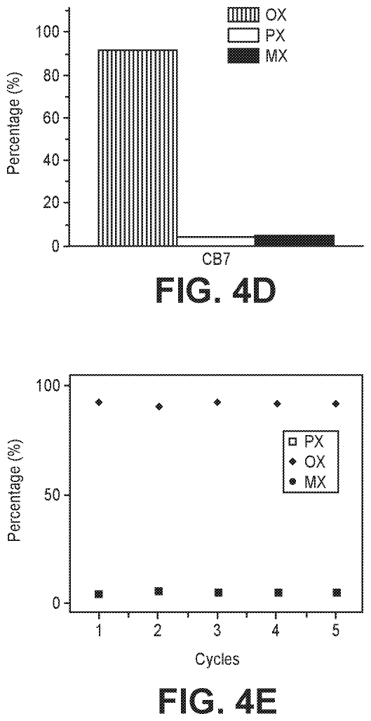

A separation process of disubstituted benzene isomers is illustrated in FIG. 4A, in accordance with one or more embodiments of the present invention. Xylene isomers have moderate solubility in water and were extracted using an aqueous solution of CB7. The separation process included dissolving CB7 in neat water to obtain an aqueous solution of CB7 (4 mg/ml) and subsequently combining xylene isomers at a 1:1:1 mixture of OX:MX:PX with the aqueous solution of CB7 in the first extraction tower. After stirring, the un-emulsified water phase was separated from the xylene phase. The ¹H NMR spectra of the water phase showed clear differences from the original aqueous solution of CB7 (FIG. 4B). In particular, the signals of H₂ and H₃ of CB7 displayed an upfield shift (Δδ=−0.03 and −0.05 ppm, respectively), implying the formation of a host-guest complex between CB7 and xylene molecules. In addition, two groups of symmetric multiple peaks, which were close to the typical signals of H_{b} and He of OX, as well as one single peak of methyl proton, were detected and exhibited remarkable upfield shifts (dashed red line area in FIG. 4B). However, the asymmetric multiple peaks for the aromatic protons (Hₑ, H_{f}, and H_{g}) of MX and the single peak for the aromatic proton (Hᵢ) of PX could not be found. These results implied that CB7 selectively complexed with OX from the xylene mixture in the aqueous phase.

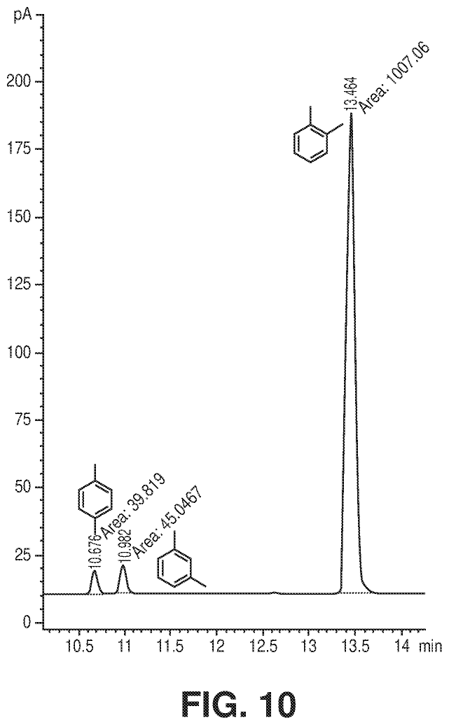

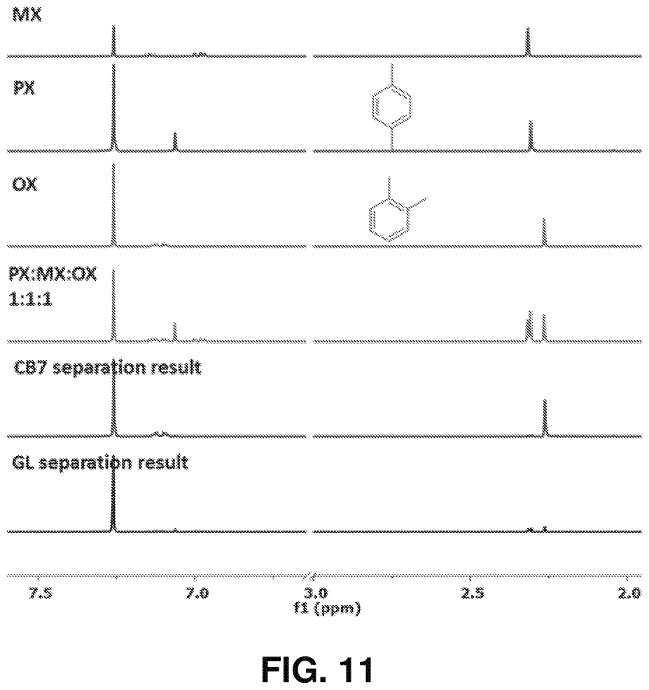

With continued reference to FIG. 4A, after the first extraction, the water phase was transferred to the second extraction tower and extracted using an organic solvent. As described above, suitable organic solvents include, without limitation, hexane, pentane, and cyclohexane, among others. For NMR experiments, CDCl₃ was selected as the organic solvent for the extraction. FIG. 4C shows the ¹H NMR spectra of xylene molecules and organic phase after the second extraction in CDCl₃. Three single peaks centered at 2.32, 2.31, and 2.26 ppm were assigned to the methyl groups of MX, PX and OX, respectively. After extraction, only the peak at 2.26 ppm (OX) could be seen in the organic phase. In view of these results, OX was selectively separated from the xylene isomers using the aqueous solution of CB7. Gas chromatograph (GC) analysis showed that the ratio or relative amounts of OX was up to 92.2% while the values of MX and PX were only 4.1% and 3.7% after the separation (FIG. 4D and FIG. 10). It was further demonstrated that the extracted aqueous solution of CB7 was able to selectively separate OX from xylene mixtures without any loss in performance after recycling 5 times (FIG. 4E). ¹H NMR illustrated the selective mechanism of extraction. For example, it was confirmed that the cavity of CB7 was responsible for the isomers separation through host-guest interactions with OX and that the glycoluril (GL) monomer units individually and separately from the cavity have minimal impact on the selectivity for OX (FIG. 11).

### Example 4

### Host-Guest Binding Experiments of Xylenes and CB7

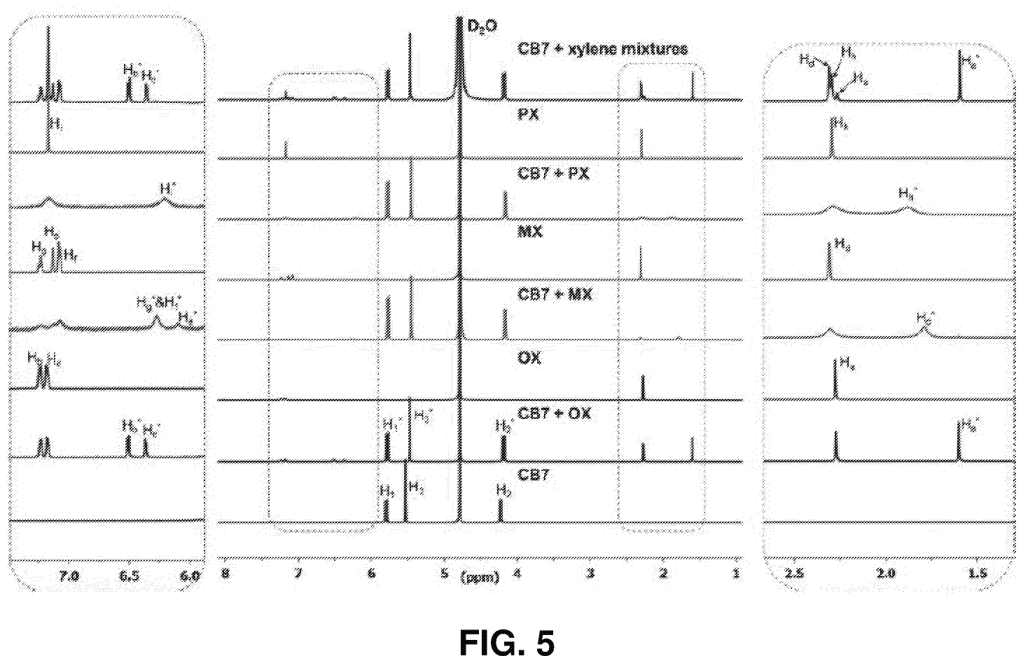

Host-guest binding between CB7 and three xylene molecules in water demonstrated the selective mechanism for isomer recognition. The host-guest binding property of CB7 and OX was tested by adding two equivalents of OX into a D₂O solution of CB7 at about 298 K and obtaining ¹H NMR spectra thereof. The ¹H NMR spectra showed distinctive differences from that of the free CB7 and OX, which demonstrated the formation of the complex OX@CB7 (FIG. 5). Both complexed and un-complexed signals of OX were observed indicating a slow-exchange complexation behavior in the NMR time-scale. The stoichiometry of the complex OX@CB7 was determined to be 1:1 by integration of the complexed signals of CB7 and OX. Moreover, the spectrum showed the complexed signals of CB7 (H₁* at 5.79-5.81 ppm, H₂* at 4.22-4.24 ppm, and H₃* at 5.53 ppm, respectively), but no un-complexed signals (H₁ at 5.77-5.79 ppm, H₂ at 4.17-4.20 ppm, and H₃ at 5.47 ppm, respectively) were detected. The complexation between CB7 and OX was thus very strong given that there was almost no free CB7 detected in ¹H NMR spectra. The binding constant of OX@CB7 was larger than 1.7×10⁵ M⁻¹ based on the assumption that the ratio of OX@CB7 to CB7 was over 20/1 according to the error associated with the NMR spectroscopic technique. Moreover, it was noticed that the methyl protons signals of PX and MX broadened when complexed with CB7, while for OX, the methyl proton signal showed a sharp peak. This difference supported the different kinetic behavior of the three guest isomers in the presence of CB7. A competition experiment was performed by adding 1.2 equivalent xylene mixtures (1:1:1, v:v:v) into CB7 aqueous solution. Only complexed OX signals (Hₐ* at 1.48 ppm, H_{b}* at 7.10 ppm, and H_{c}* at 7.15 ppm, respectively) appeared in the ¹H NMR spectra. By contrast, complexed signals of PX@CB7 and MX@CB7 were not observed.

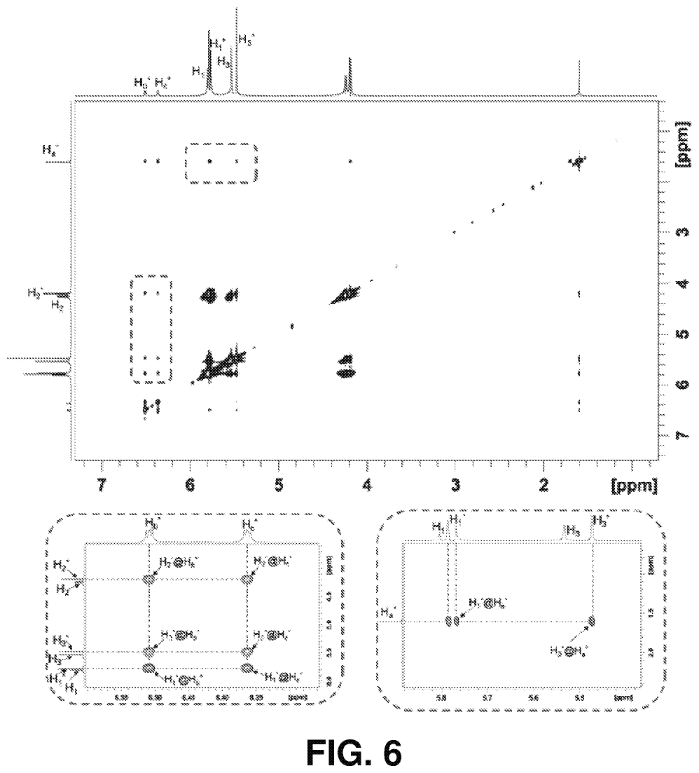

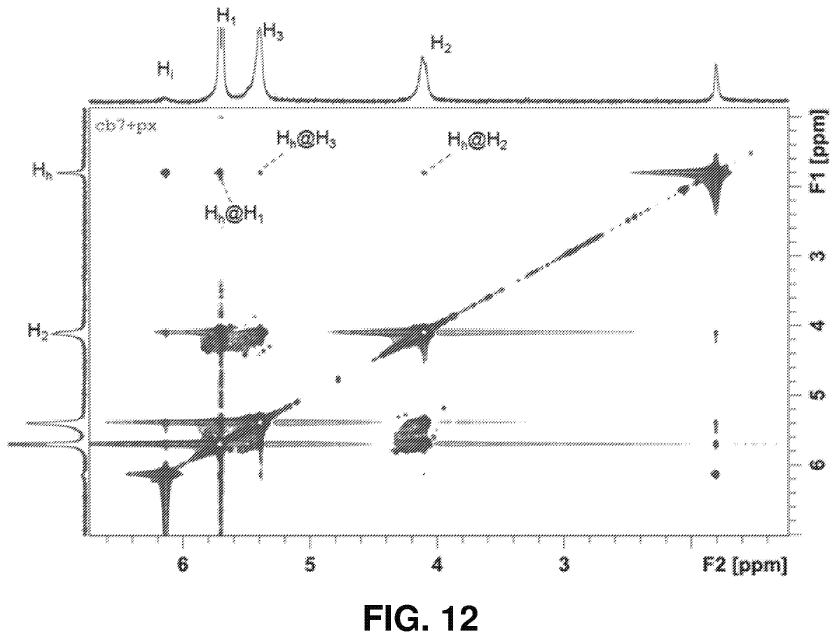

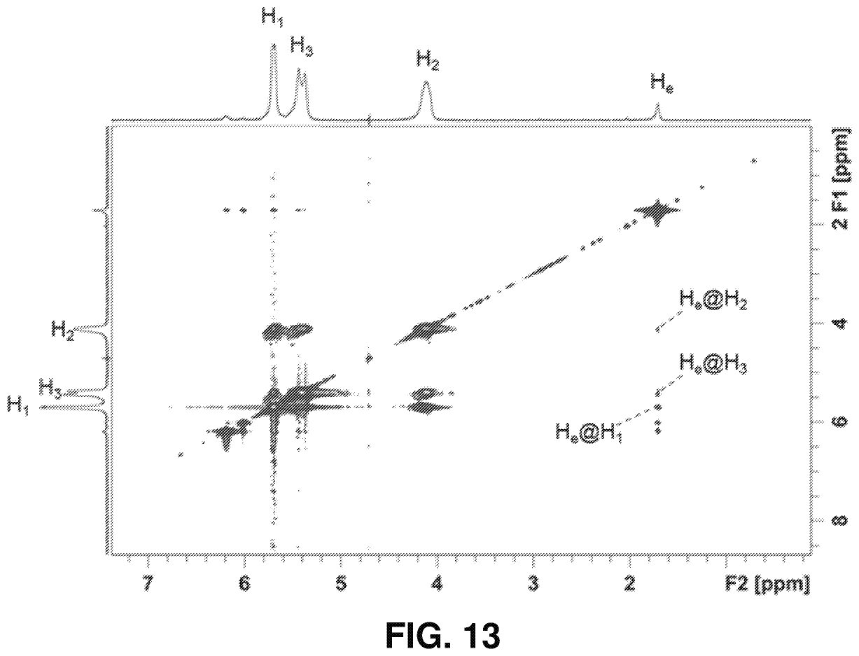

In some embodiments, 2D NOESY spectroscopic characterization provides information regarding the relative location of CB7 and xylene isomers in the complexes. For example, as shown in FIG. 6, cross-peak signals were detected between the proton Hₐ*, H_{b}* and H_{c}* of complexed OX and protons H₁*, H₂* and H₃* of the complexed CB7 indicating that OX threaded through the central cavity of CB7 to form a 1:1 complex. Under the same conditions, PX and MX showed similar complexation behavior with CB7 (FIGS. 12-13).

### Example 5

### Thermodynamic Parameters of CB7 and Xylenes

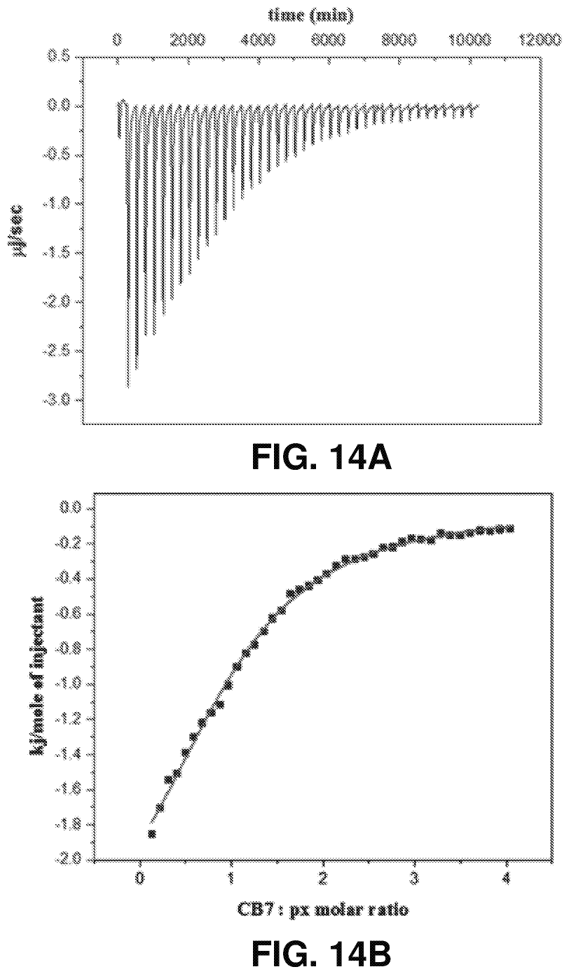

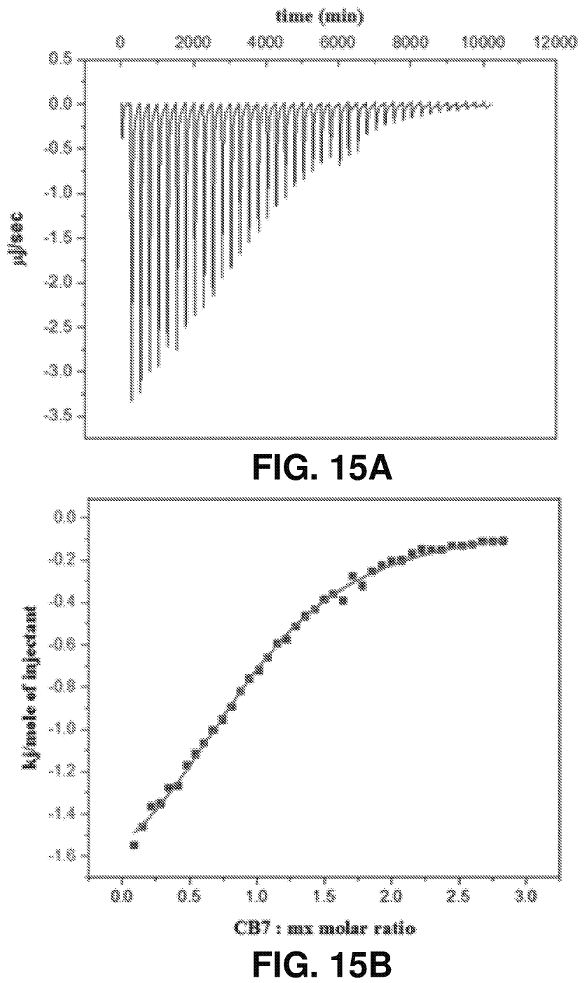

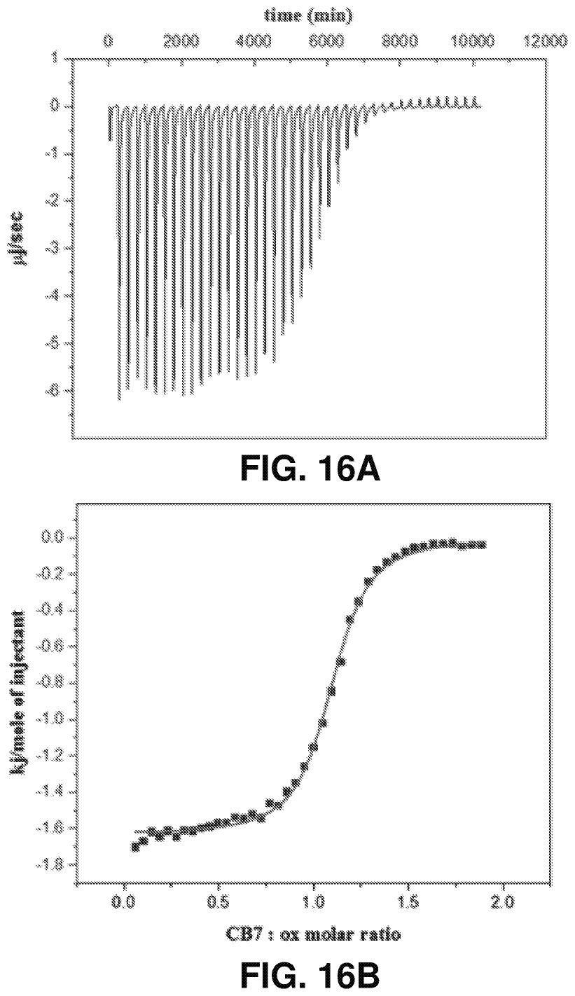

The thermodynamic parameters of CB7 and xylenes can be used to provide estimates of the binding constant. For example, to estimate the binding constant Kₐ of the complexation between CB7 and xylene isomers, isothermal titration calorimetry (ITC) experiments can be conducted in aqueous solution. Due to the limited solubility of xylene molecules in water, CB7 was used as titrant and xylene molecules were used as titrates (FIGS. 14-16). All three xylene isomers showed 1:1 binding stoichiometry with CB7, which was consistent with the NMR experiment results. The binding constant Kₐ of the complex OX@CB7, MX@CB7, and PX@CB7 was calculated to be 8.9×10⁵ M⁻¹, 4.2×10⁴ M⁻¹ and 3.3×10⁴ M⁻¹ (Table 1), demonstrating that the binding constant of OX@CB7 was much higher than that of MX@CB7 (about 21 times) and PX@CB7 (about 27 times).

Thermodynamic analysis demonstrated that the formation of xylene@CB7 complexes was enthalpy driven. The large favorable binding enthalpic gain corresponded to the formation of inclusion complexes and release of high-energy water molecules inside the cavity of CB7, which compensated for the unfavorable binding entropic contribution. Compared to MX and PX, OX was more entropically disfavored (TAS=−11.9, −10.4, and −10.8 kj mol⁻¹ for OX, MX, and PX, respectively), but apparently the higher enthalpic value (ΔH=−45.9, −36.7, and −36.6 kj mol⁻¹ for OX, MX, and PX, respectively) dictated the binding with CB7.

### Example 6

### Kinetic Analysis of the Host-Guest Exchange Process

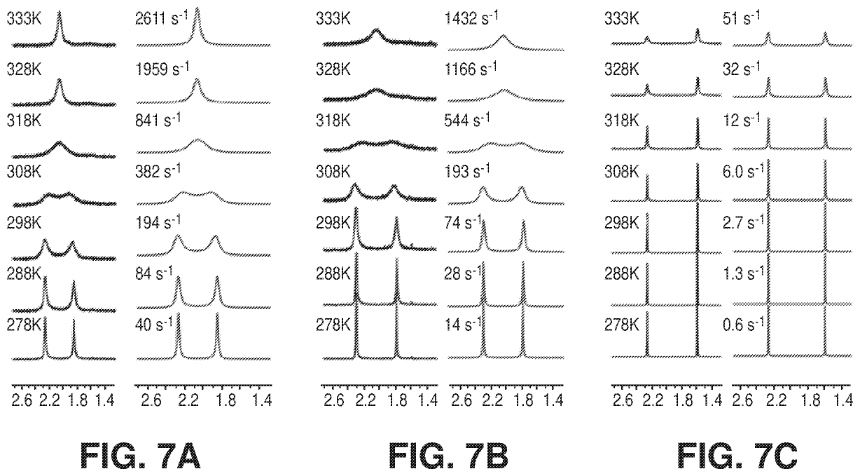

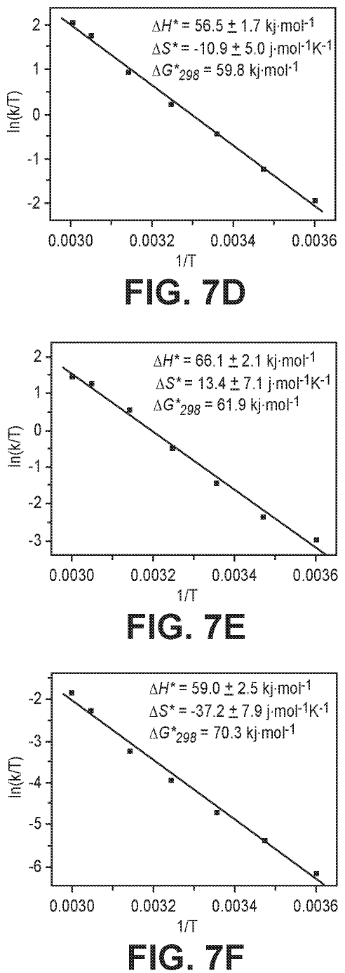

A kinetic analysis of the host-guest exchange process was conducted. In consideration of the slow exchange complexation process between CB7 and xylene molecules, variable-temperature ¹H NMR experiments were performed to record the kinetic and activation parameters of the complexes in D₂O. The rate constants for decomplexation, namely the first order rate constant for decomplexation process of the complexes CB7@xylenes, were calculated by line shape analysis at a series of temperatures. The results are listed in FIG. 7A-7F and Table 1. At 298K, the rate constants for decomplexation of OX@CB7, MX@CB7 and PX@CB7 were 194 s⁻¹, 74 s⁻¹, and 2.7 s⁻¹, respectively, indicating the decomplexation process of CB7@OX was much slower than the other two. The complexation rate constants of OX@CB7, MX@CB7 and PX@CB7 obtained by k₁=Kₐ/k₋₁ were 2.4×10⁶ M⁻¹ s⁻¹, 3.1×10⁶ M⁻¹ s⁻¹, and 6.4×10⁶ M⁻¹ s⁻¹, respectively, showed that OX had the slowest complexation rate with CB7 compared to PX and MX. The binding ability between CB7 and the three xylene molecules appeared to be mainly controlled by their distinctive rate constants of decomplexation. In addition, the activation enthalpy (ΔH≠), entropy (ΔS≠) and energy (ΔG≠) at 298K were also obtained from Eyring plots. For OX@CB7, MX@CB7, and PX@CB7, ΔH≠ were 59.0, 66.1, 56.5 kJ mol⁻¹ and ΔS≠ were −37.2, 13.4, −10.9 kJ mol⁻¹, respectively. The much lower activation enthalpy ΔS≠ of OX@CB7 resulted in higher activation energy ΔG≠ (70.3 kJ mol⁻¹ for OX@CB7) which was consistent with k₋₁ values calculated by dynamic NMR analysis.

### Example 7

### Shape-Sorting Effect

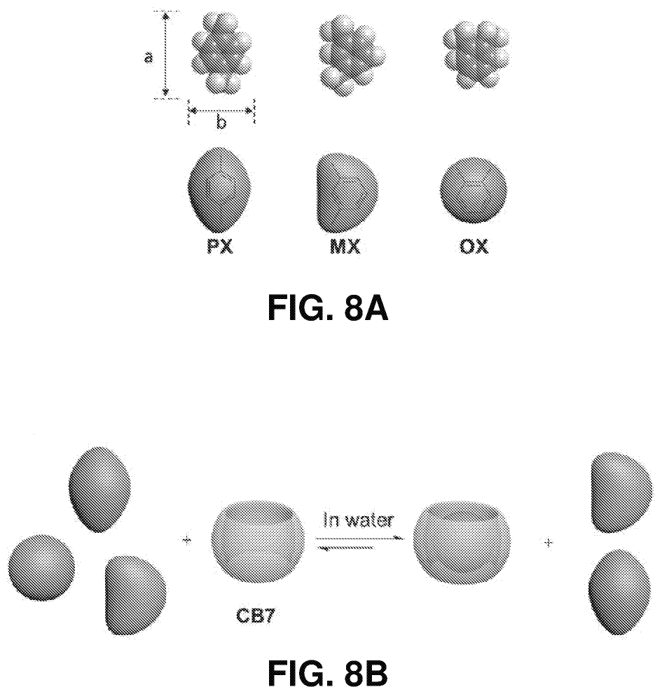

The binding affinity was determined by considering the packing coefficients (PC, volume of the guest divided by the volume of the host cavity), matching Rebek's 55% empirical solution for ideal host-guest complex. However, xylene isomers possess nearly uniform molecular volume, so the selectivity was not explained well by packing coefficients (Table 2). Because of the different substituted positions, xylene isomers have a different aspect ratio a/b (FIGS. 8A-8B). As shown in Table 2, the aspect ratio was determined to be 1.4 for PX, 1.2 for MX, and 1.0 for OX, indicating OX had the smallest aspect ratio (discoid shape) and perhaps a better fit for the spherical cavity of CB7. Accordingly, the selectivity could be at least partially explained by a shape-sorting effect.

### Example 8

### DFT Calculations

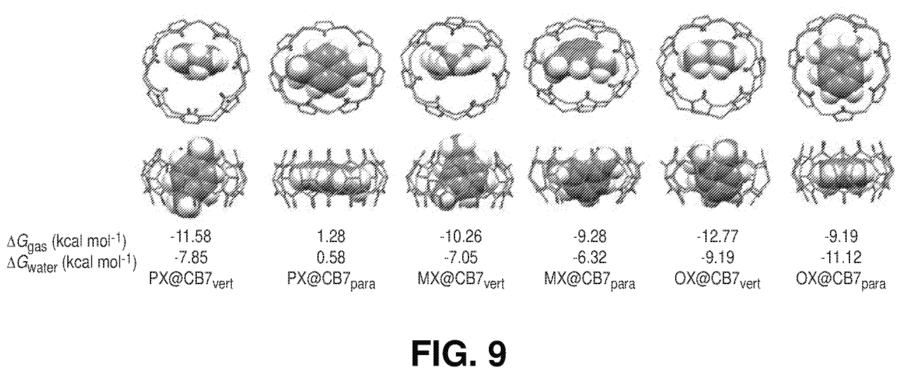

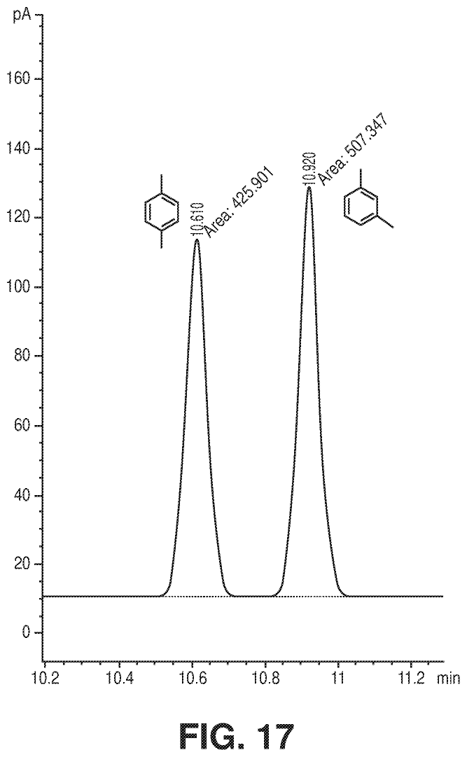

DFT calculations were conducted. FIG. 9 shows the DFT-optimized structures of the corresponding inclusion complexes xylenes @CB7, explaining the pore size and cavity shape matching of the xylene isomers. For each inclusion, according to the orientation where the guest may enter the cavity, two possible complex modes were designed, namely a vertical complex and parallel complex. In the gas phase, all the three xylene molecules tended to form vertical complexes. The binding energies were about −11.58, −10.26, and −12.77 kcal mot′ for vertical complex PX@CB7ᵥₑᵣₜ, MX@CB7ᵥₑᵣₜ, and OX@CB7ᵥₑᵣₜ, respectively. The OX@CB7ᵥₑᵣₜ possessed the lowest binding energy which was slightly different from the other two vertical complexes. However, in water phase, the results were quite different. PX and MX tended to form vertical complexes due to their spindle structures. However, the parallel inclusion of OX in OX@CB7ₚₐᵣₐ had lower binding energy than its vertical mode (−12.77 vs −11.12 kcal/mol⁻¹), indicating that OX preferred to form parallel inclusion complex with CB7 in water. Furthermore, the binding energy of OX@CB7ₚₐᵣₐ (−11.12 kcal mol⁻¹) was much lower than that of PX@CB7ᵥₑᵣₜ (−7.85 kcal mol⁻¹) and MX@CB7ᵥₑᵣₜ (−7.05 kcal mol⁻¹). Accordingly, the discoid structure of OX better matched the cavity of CB7 in water. PX and MX, due to the similar vertical complex mode, had close binding energies with CB7 and distinct aspect ratios (1.4 for PX and 1.2 for MX). This result at least partially explained why CB7 did not show obvious separation ability for PX/MX (FIG. 17).

### Example 9

### Separation of Other Disubstituted Benzene Isomers

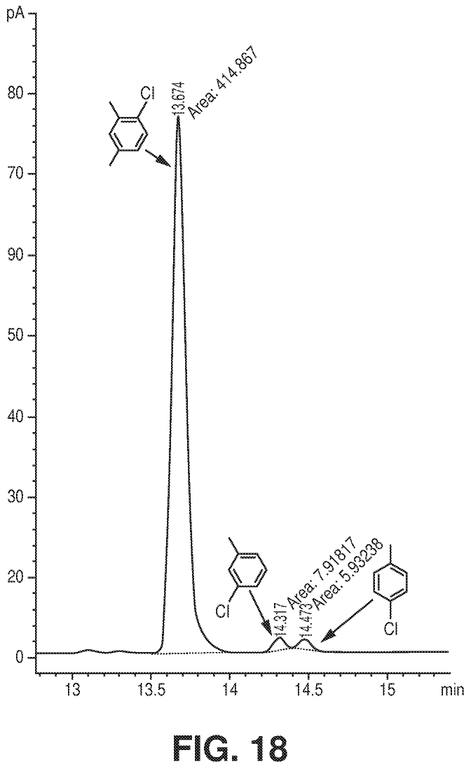

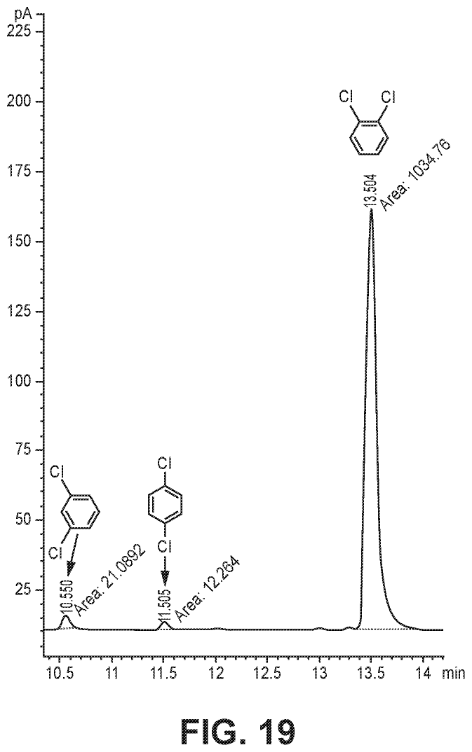

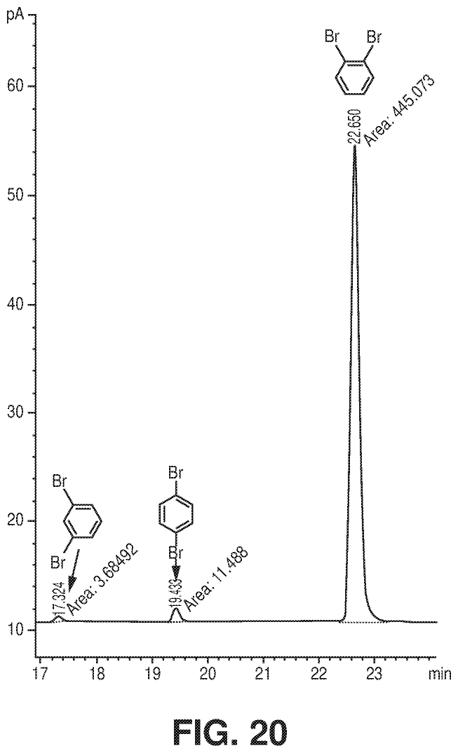

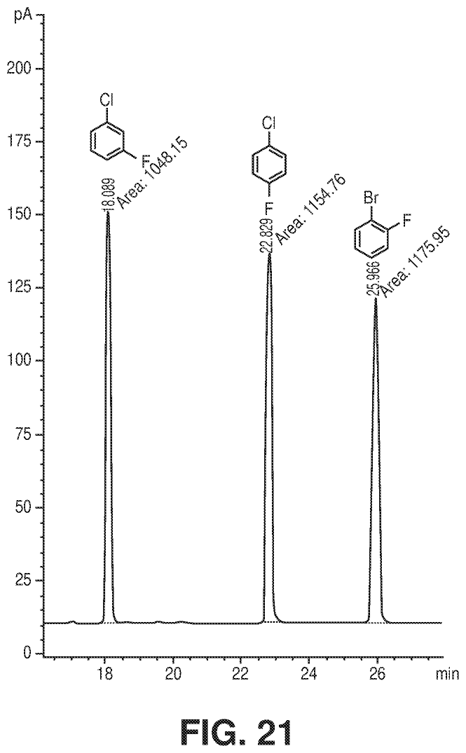

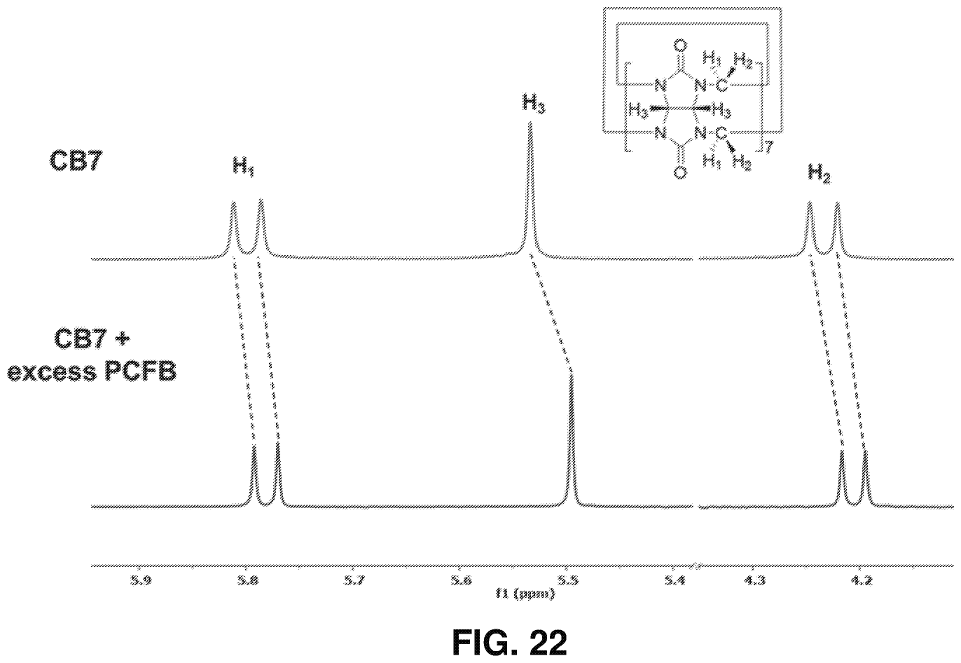

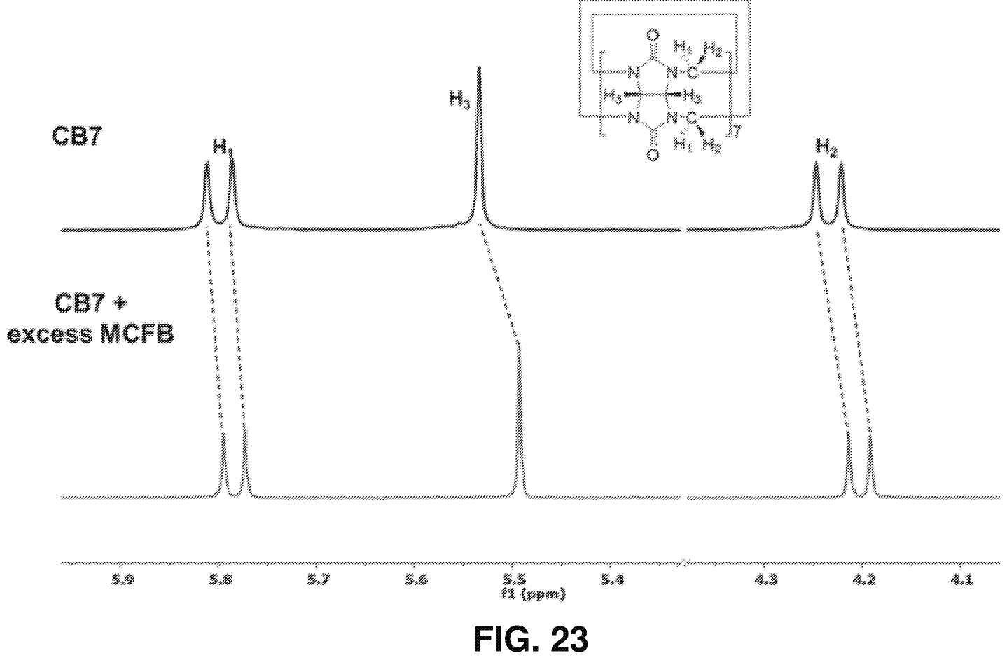

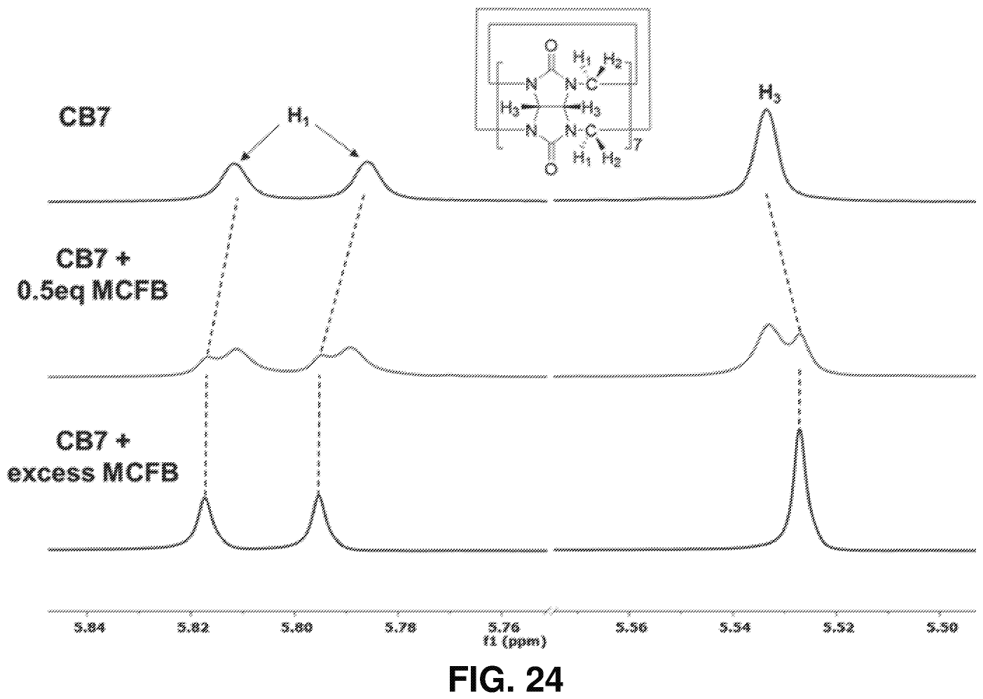

Separations involving other disubstituted benzene isomers were conducted, including dichlorobenzenes (DCB), dibromobenzenes (DBB), chlorotoluenes (CT), and chlorofluorobenzene (CFB) isomers. As expected, the results showed the ortho-substituted isomers were easily separated from their counterparts, with the separation efficiency being about 96.7% for O-DBB, 96.9% for O-DCB, and 96.8% for 0-CT in one extraction cycle (Table 3 and FIGS. 18-21). Again, the success of these selective separations was attributed to their distinct aspect ratios (O-DBB, O-DCB, and O-CT had the same aspect ratio of 1.1), which was consistent with the proposed shape fitting effect. However, in the case of CFB, the separation efficiency was 34.2%, 31.0%, and 34.8% for P-CFB, M-CFB and O-CFB. Due to the small PC values (volume of the CFB isomers divided by the volume of CB7 cavity), the host-guest experiments between CFB isomers and CB7 were performed (FIGS. 22-24). The result illustrated that all three CFB isomers could form complexes with CB7 with pore selectivity, which was likely due to the comparable size of fluorine and hydrogen, which further supported the enhanced molecular recognition of the host CB7.

TABLE 1
Thermodynamic and Kinetic Parameters of Different
Xylene Isomers in CB7 at 298K in Water

| | Kₐ | k₋₁^{a} | k₁ | ΔH | TΔS | ΔG₂₉₈^{K} | ΔH^{≠} | ΔS^{≠} |
|---|---|---|---|---|---|---|---|---|
| | (M⁻¹) | (s⁻¹) | (M⁻¹s⁻¹) | (kj mol⁻¹) | (kj mol⁻¹) | (kj mol⁻¹) | (kj mol⁻¹) | (j K⁻¹ mol⁻¹) |
| PX | 3.3 × 10 [symbol] | 194 | 6.4 × 10⁶ | −36.6 | −10.8 | 59.8 | 56.5 | −10.9 |
| MX | 4.2 × 10 [symbol] | 74 | 3.1 × 10⁶ | −36.7 | −10.4 | 61.9 | 66.1 | 13.4 |
| OX | 8.9 × 10 [symbol] | 2.7 | 2.4 × 10⁴ | −45.9 | −11.9 | 70.3 | 59.0 | −37.2 |

^{a}Values from Dynamic NMR calculation. [symbol]
indicates data missing or illegible when filed

TABLE 2
Molecular Size of Xylene Isomers

| | a | b | | Volume^{a} | PC^{b} |
|---|---|---|---|---|---|
| | (Å) | (Å) | a/b | (Å3) | (%) |
| PX | 9.2 | 6.6 | 1.4 | 118 | 49 |
| MX | 8.9 | 7.3 | 1.2 | 118 | 49 |
| OX | 7.6 | 7.5 | 1.0 | 118 | 49 |

^{a}Volumes calculated by HyperChem software.
^{b}Packing coefficient obtained by dividing the volume of xylene molecules by the inner cavity volume of CB7 (242 Å, see ref. 38).

TABLE 3
Molecular Size and Relative Amount of the Disubstituted
Benzene Isomers after the Second Extraction

| | a | b | | Volume | PC | Relative |
|---|---|---|---|---|---|---|
| | (Å) | (Å) | a/b | (Å³) | (%) | amount (%)^{a} |
| p-Dibromobenzene(P-DBB) | 10.2 | 6.7 | 1.5 | 127 | 53 | 2.5 |
| m-Dibromobenzene(M-DBB) | 9.3 | 7.3 | 1.3 | 128 | 53 | 0.8 |
| G-Dichlorobenzene(O-DBB) | 8.0 | 7 2 | 1.1 | 127 | 52 | 96.7 |
| p-Dichlorobenzene(P-DCB) | 9.6 | 6.7 | 1.4 | 113 | 47 | 1.1 |
| m-Dichlorobenzene-DCB) | 8.8 | 7.3 | 1.2 | 113 | 47 | 2.0 |
| o-Dichlorobenzene(O-DCB) | 7.7 | 7.3 | 1.1 | 113 | 47 | 96.9 |
| p- Chlorotolene(P-CT) | 9.4 | 6.6 | 1.4 | 116 | 48 | 1.4 |
| m-Chlorotolene (M-CT) | 9.0 | 7.3 | 1.2 | 116 | 48 | 1.8 |
| o-Chlorotolene (O-CT) | 7.9 | 7.3 | 1.1 | 115 | 48 | 96.8 |
| p-chlorofluorobenzene(P-CFB) | 9.0 | 6.7 | 1.3 | 101 | 42 | 34.2 |
| m-chlorofluorobenzene(M-CFB) | 8.5 | 7.1 | 1.2 | 101 | 42 | 31.0 |
| o-chlorofluorobenzene(O-CFB) | 8.5 | 7.1 | 1.2 | 101 | 42 | 34.8 |

^{a}Results got from GC analysis after the separation of 1:1:1 jinxed disubstituted benzene isomers. See supplemental information.

### Example 10

### Separation of Trimethylbenzene (TMB) Isomers

Trimethylbenzene (TMB) isomers, 1,2,3-trimethylbenzene (123TMB), 1,2,4-trimethylbenzene (124TMB) and 1,3,5-trimethylbenzene (135TMB), are important chemical feedstocks in petrochemical and pharmaceutical industries. CB7 was used for successive separation of three trimethylbenzene (TMB) isomers with more than 96% specificity. A two-steps liquid-liquid extraction method was used for the separation experiment. In order to detect the changes of the relative amount of these three TMB isomers, a carrier solvent 1,3,5-triisopropylbenzene (TIPB) whose size excluded from the pores of CB7 was used for the separation process.

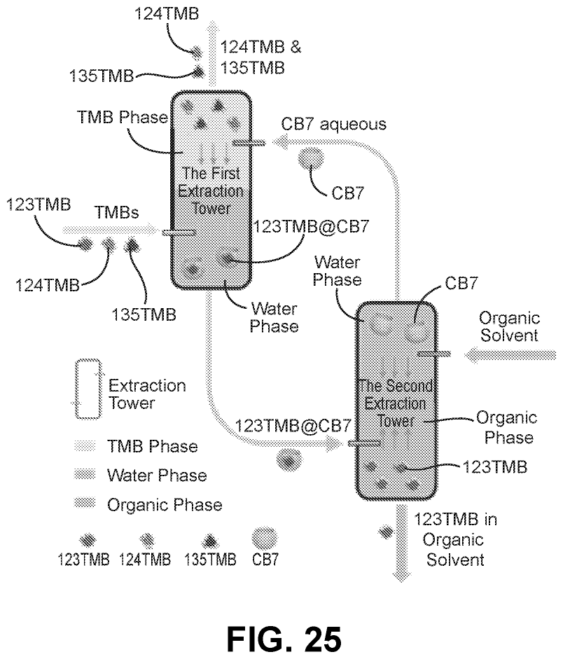

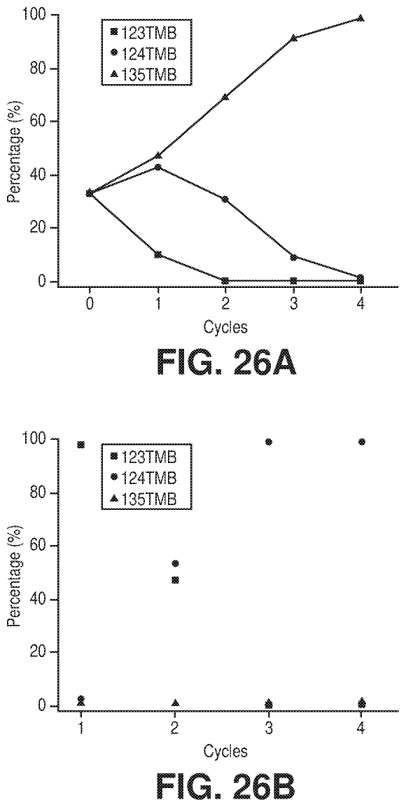

As shown in FIG. 25, a CB7 aqueous solution (4 mg/ml) and 1:1:1 (v:v:v) TMB mixtures were mixed together and added to the first extraction tower. After stirring for 30 minutes, a water phase and TMB phase was separated and the TMB phase was detected by GC analysis. The results showed the ratio of 123TMB decreased from 33% to 12% (FIG. 26A). Then the water phase was filtered and transferred to the second extraction tower and extracted by organic solvent. GC result of the organic phase shows that the ratio of 123TMB was more than 97% (FIG. 26B). The extracted CB7 aqueous solution was used for the second extraction cycle. As we can see in FIG. 26A, there were only 124TMB (36%) and 135TMB (64%) in the TMB phase. For the organic phase in FIG. 26B, it was discovered that CB7 extracted both 123TMB (47%) and 124TMB (53%). When the third extraction cycle was repeated, it was discovered that CB7 only extracted 124TMB. The ratio of 124TMB in organic phase was more than 98%. These separation experiments clearly illustrate the successive separation of TMB isomers by CB7.

1. A process of separating aromatic isomers comprising:
contacting an isomers solution including one or more aromatic isomers, with an aqueous solution including a cucurbituril macrocycle, to produce a first aqueous phase and a first organic phase, wherein the cucurbituril macrocycle is selective for the extraction of at least one of said aromatic isomers.

2. The process according to claim 1, wherein the one or more aromatic isomers include one or more benzene isomers.

3. The process of claim 1, wherein the one or more aromatic isomers includes one or more of an ortho-substituted aromatic isomer, a meta-substituted aromatic isomer, and a para-substituted aromatic isomer.

4. The process according to claim 3, wherein the cucurbituril macrocycle is selective for the extraction of the ortho-substituted aromatic isomer.

5. The process of claim 1, wherein the isomers solution includes at least one of the following (1) to (14):
(1) one or more of o-xylene, m-xylene, p-xylene, and ethylbenzene;
(2) one or more of o-dibromobenzene, m-dibromobenzene and p-dibromobenzene;
(3) one or more of o-dichlorobenzene, m-dichlorobenzene, and p-dichlorobenzene;
(4) one or more of o-bromotoluene, m-bromotoluene and p-bromotoluene;
(5) one or more of o-chlorotoluene, m-chlorotoluene, and p-chlorotoluene;
(6) one or more of 1-bromo-2-chlorobenzene, 1-bromo-3-chlorobenzene, and 1-bromo-4-chlorobenzene;
(7) one or more of 2-chlorophenol, 3-chlorophenol, and 4-chlorophenol;
(8) one or more of o-phenylenediamine, m-phenylenediamine, and p-phenylenediamine;
(9) one or more of o-xylylenediamine, m-xylylenediamine, and p-xylylenediamine;
(10) one or more of o-diethylbenzene, m-diethylbenzene, and p-diethylbenzene;
(11) one or more of o-ethyltoluene, m-ethyltoluene, and p-ethyltoluene;
(12) one or more of 1,2,3-trimethylbenzene (hemimellitene), 1,2,4-trimethylbenzene (pseudocumene), and 1,3,5-trimethylbenzene (mesitylene);
(13) one or more of 1,2,4,5-tetramethylbenzene (durene), 1,2,3,5-tetramethylbenzene tetramethylbenzene (isodurene), and 1,2,3,4-tetramethylbenzene (prehnitene); and
(14) one or more of 1,4-diethylbenzene (para-diethylbenzene), 1,3-diethylbenzene (meta-diethylbenzene), and 1,2-diethylbenzene (ortho-diethylbenzene).

6. The process according to claim 5, wherein the cucurbituril macrocycle is selective for the extraction of one or more of o-xylene, o-dibromobenzene, o-dichlorobenzene, o-bromotoluene, o-chlorotoluene, 1-bromo-2-chlorobenzene, 2-chlorophenol, o-phenylenediamine, o-xylylenediamine, o-diethylbenzene, 1,2,3-trimethylbenzene, 1,2-diethylbenzene (ortho-diethylbenzene), 1,2,4,5-tetramethylbenzene (durene), 1,2,3,5-tetramethylbenzene tetramethylbenzene (isodurene), and 1,2,3,4-tetramethylbenzene (prehnitene).

7. The process of claim 1, wherein the cucurbituril macrocycle has the following chemical structure: 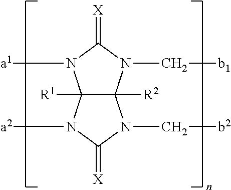
wherein:
a¹ indicates the point of attachment to b¹;
a² indicates the point of attachment to b²;
n is 1-20;
X is O, S, or NH; and
R¹ and R² are independently selected from the group consisting of hydrogen, H, optionally substituted C₁-C₃₀ alkyl group; optionally substituted C₂-C₃₀ alkenyl group; optionally substituted C₂-C₃₀ alkynyl group; optionally substituted C₂-C₃₀ carbonylalkyl group; optionally substituted C₁-C₃₀ thioalkyl group; optionally substituted C₁-C₃₀ alkylthiol group; optionally substituted C₁-C₃₀ hydroxyalkyl group; optionally substituted C₁-C₃₀ alkylsilyl group; optionally substituted C₁-C₃₀ aminoalkyl group; optionally substituted C₁-C₃₀ aminoalkylthioalkyl group; optionally substituted C₅-C₃₀ cycloalkyl group; optionally substituted C₂-C₃₀ heterocycloalkyl group; optionally substituted C₆-C₃₀ aryl group; optionally substituted C₆-C₃₀ arylalkyl group; optionally substituted C₄-C₃₀ heteroaryl group; and optionally substituted C₄-C₃₀ heteroarylalkyl group.

8. The process according to claim 7, wherein n is 7.

9. The process of claim 7, wherein X is O.

10. The process of claim 7, wherein R¹ and R² are H.

11. The process of claim 1, wherein contacting the isomers solution with the aqueous solution causes at least a portion of at least one of said aromatic isomers to be transferred from the isomers solution to the aqueous solution.

12. The process according to claim 11, wherein the portion of at least one of said aromatic isomers is transferred from the isomers solution to the aqueous solution through the formation of a host-guest complex in which the cucurbituril macrocycle is the host and the transferred aromatic isomer is the guest.

13. The process according to claim 12, wherein the first aqueous phase includes the host-guest complex; and wherein the first organic phase includes the isomers solution, the isomers solution having a reduced concentration of the transferred aromatic isomer.

14. The process according to claim 13, further comprising recovering at least a portion of aromatic isomer from the first aqueous phase, using an organic solution, to produce a second aqueous phase and a second organic phase.

15. The process according to claim 14, wherein the second aqueous phase includes at least a portion of the cucurbituril macrocycle from the first aqueous phase, and wherein the second organic phase includes the portion of the aromatic isomer recovered from the first aqueous phase.

16. The process according to claim 15, further comprising recycling the second aqueous phase for use in one or more separation cycles.

17. A liquid-liquid extraction solvent comprising: an aqueous solution of a cucurbituril macrocycle, wherein the cucurbituril macrocycle is selective for the extraction of at least one aromatic isomer and wherein the cucurbituril has the following chemical structure: 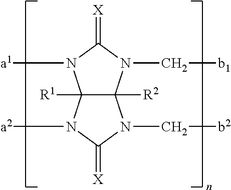
wherein:
a¹ indicates the point of attachment to b¹;
a² indicates the point of attachment to b²;
n is 1-20;
X is O, S, or NH; and
R¹ and R² are independently selected from the group consisting of hydrogen, H, optionally substituted C₁-C₃₀ alkyl group; optionally substituted C₂-C₃₀ alkenyl group; optionally substituted C₂-C₃₀ alkynyl group; optionally substituted C₂-C₃₀ carbonylalkyl group; optionally substituted C₁-C₃₀ thioalkyl group; optionally substituted C₁-C₃₀ alkylthiol group; optionally substituted C₁-C₃₀ hydroxyalkyl group; optionally substituted C₁-C₃₀ alkylsilyl group; optionally substituted C₁-C₃₀ aminoalkyl group; optionally substituted C₁-C₃₀ aminoalkylthioalkyl group; optionally substituted C₅-C₃₀ cycloalkyl group; optionally substituted C₂-C₃₀ heterocycloalkyl group; optionally substituted C₆-C₃₀ aryl group; optionally substituted C₆-C₃₀ arylalkyl group; optionally substituted C₄-C₃₀ heteroaryl group; and optionally substituted C₄-C₃₀ heteroarylalkyl group.

18. The process according to claim 17, wherein n is 7.

19. The process of claim 17, wherein X is O.

20. The process of claim 17, wherein R1 and R2 are H.
